(19) United States
(12) Patent Application Publication
(10) Pub. No.: US 2026/0174424 A1
(43) Pub. Date: Jun. 25, 2026

(54) SUTURE ANCHOR ASSEMBLY

(71) Applicant: Arthrex, Inc., Naples, FL (US)

(72) Inventors: Eric ZAJAC, Naples, FL (US); Tom DOONEY, Naples, FL (US); Derek SULLIVAN, Naples, FL (US); Patrick SMITH, St. Petersburg, FL (US); Nathanael GAMSO, Bonita Springs, FL (US)

(21) Appl. No.: 19/421,811

(22) Filed: Dec. 16, 2025

Related U.S. Application Data
(60) Provisional application No. 63/736,408, filed on Dec. 19, 2024.
(60) Provisional application No. 63/850,846, filed on Jul. 25, 2025.
(60) Provisional application No. 63/894,768, filed on Oct. 7, 2025.

(51) Int. Cl. A61B 17/04

(52) U.S. Cl. CPC A61B 17/0401; A61B 2017/0412; A61B 2017/0414; A61B 2017/0424; A61B 2017/0425; A61B 2017/0427; A61B 2017/044; A61B 2017/0446

(57) ABSTRACT

An anchor assembly secures a tissue to a bone with a repair strand. The anchor assembly includes an anchor sleeve having a lumen and an inner thread, an anchor tip having an opening, and an anchor insert inserted into the lumen of the anchor sleeve. The anchor sleeve is inserted into a bone socket in the bone. The opening receives the repair strand. The anchor insert has an outer thread configured to engage the inner thread of the anchor sleeve, where the anchor insert rotates relative to the anchor tip while being inserted into the anchor sleeve. The anchor assembly is used in a method of securing tissue to bone.

### CROSS REFERENCE TO RELATED APPLICATIONS

This application claims the benefit of U.S. Provisional Patent App. No. 63/736,408, filed Dec. 19, 2024, U.S. Provisional Patent App. No. 63/850,846, filed Jul. 25, 2025, and U.S. Provisional Patent App. No. 63/894,768, filed Oct. 7, 2025, the disclosures of which is hereby incorporated by reference herein.

### TECHNICAL FIELD

The disclosure relates to the field of surgery and, more specifically, to anchors and methods of anchoring repair strands.

### BACKGROUND

Repair or reattachment of tissue to bone is often performed by suturing. The procedure typically requires the surgeon to pass suture material through the tissue and to anchor the suture to the bone.

### SUMMARY

The present inventors recognize that knot tying during surgery can be tedious and time-consuming. Knots have a tendency to deform or collapse as the surgeon manually forces the knots down into the proper position. The knots and sutures themselves are also often exposed to abrasion or cutting by sharp or rough areas along the walls of the bone canal into which anchors are typically inserted to provide fixation of tendon to bone.

Furthermore, the present inventors recognize that variable bone density is present amongst the patient population. Some younger, healthier patients have hard, dense bone that allows for threaded suture anchors to gain secure purchase in the bone. On the other hand, bone in older patients may have reduced density and hardness that can cause less optimal fixation strength or failure of a repair. In these cases, a larger anchor with additional fixation features may be necessary to gain secure fixation. Even in cases when anchor fixation is achieved, the surrounding bone may not be sufficient to support tension of repair strands resulting in the repair strand cutting through bone or the bone socket widening.

Thus, a first aspect of the present disclosure is directed to an anchor assembly configured to secure a tissue to a bone with a repair strand. The anchor assembly includes an anchor sleeve having a lumen, an anchor tip having an opening, and an anchor insert inserted into the lumen of the anchor sleeve. The anchor sleeve is inserted into a bone socket in the bone. The opening receives the repair strand. The anchor assembly is used in a method of securing tissue to bone.

The anchor assembly may also include an anchor sleeve that may include a plurality of wings configured to radially expand and an inner thread. The anchor sleeve may define a lumen, and the anchor sleeve may be configured to be inserted into a bone socket in the bone. The assembly also includes an anchor tip having an opening, where the opening is configured to receive the repair strand. The assembly also includes an anchor insert configured to be inserted into the lumen of the anchor sleeve. The anchor insert may have an outer thread configured to engage the inner thread of the anchor sleeve, and the anchor insert may be configured to radially expand the plurality of wings of the anchor sleeve while the anchor insert is inserted into the lumen of the anchor sleeve with the repair strand between the anchor insert and the anchor sleeve.

The anchor insert may be configured to rotate relative to the anchor tip while being inserted into the anchor sleeve. The anchor insert may be proximal of the anchor tip along a drive shaft. A proximal portion of the anchor sleeve may have a shoulder. The shoulder may extend radially from at least part of the circumference of the anchor sleeve. The anchor sleeve may have a plurality of teeth distal of the shoulder and configured to reduce rotation of the anchor sleeve relative to the bone. The shoulder may have a proximal surface with a recess configured to receive the repair strand. The anchor sleeve may have an inner surface with at least one longitudinal channel configured to receive the repair strand. The at least one longitudinal channel may interrupt the inner thread. The at least one longitudinal channel may be at least partially in a common plane as the recess. The at least one longitudinal channel includes a first longitudinal channel and a second longitudinal channel opposite the first longitudinal channel. The anchor insert may be sized to be inserted into the anchor sleeve in the absence of the repair strand without expanding the anchor sleeve. An outer width or diameter of the anchor insert may be substantially equal to or less than an inner width or diameter of the anchor sleeve. The plurality of wings may have at least one barb. The shoulder may have a lip extending radially of a tubular body. The lip may extend further radially than a portion of the shoulder. A proximal surface of the lip may be recessed from a proximal surface of the shoulder. The proximal surface of the lip may be substantially flat. The lip may have a raised protrusion forming a first recess and a second recess.

The method also includes inserting an anchor sleeve into a bone socket of a bone, where the anchor sleeve has an inner thread. The method also includes securing a repair strand to a tissue. The method also includes inserting the repair strand into an opening of an anchor tip. The method also includes inserting the anchor tip into the bone socket through the anchor sleeve. The method also includes rotating an anchor insert relative to the anchor tip to engage an outer thread of the anchor insert to the inner thread of the anchor sleeve and thereby expand the anchor sleeve.

Inserting the anchor sleeve into the bone socket of the bone may include seating a shoulder of the anchor sleeve on an outer surface of the bone. Seating the shoulder of the anchor sleeve on the outer surface of the bone may include engaging a plurality of teeth extending distally from the shoulder with the outer surface of the bone to reduce rotation of the anchor sleeve within the bone socket. The method may include receiving the repair strand between the anchor insert and the anchor sleeve, and expanding the anchor sleeve is based on a composite thickness of the anchor insert and the repair strand. Receiving the repair strand between the anchor insert and the anchor sleeve may be in a longitudinal channel on an inner surface of the anchor sleeve. The method may include expanding a plurality of wings of the anchor sleeve. Inserting the anchor tip into the bone socket may be with a drive shaft, and rotating the anchor insert may be with the drive shaft. The method may include securing a second repair strand to the tissue, inserting the second repair strand into the opening of the anchor tip, and receiving the second repair strand in the recess on the proximal surface of the shoulder. The method may include supporting the repair strand on a lip of the shoulder. The method may include securing the tissue to the lip with a second repair strand. Securing the tissue to the lip may include looping the second repair strand through the tissue and passing the second repair strand through an opening in the lip. Passing the second repair strand through the opening in the lip may be by passing the second repair strand through a loop of a suture threader and pulling the suture threader through the opening. The tissue may be secured to the second repair strand before the repair strand is inserted into the opening of the anchor tip. The method may include passing an anchor secured to the repair strand through the tissue, wherein the tissue is secured to the second repair strand before the anchor secured to the repair strand is passed through the tissue.

A second aspect of the present disclosure is directed to an anchor sleeve. The anchor sleeve may include a tubular body configured to be inserted into a socket of a bone, wherein the tubular body has a lumen configured to receive an anchor insert, and the tubular body has a plurality of wings configured to expand; and a shoulder extending at least partially around the lumen, wherein the shoulder is configured to be seated on an outer surface of the bone, the shoulder has a lip extending radially of the tubular body, and the shoulder is configured to support a repair strand extending from a tissue and through the lumen of the tubular body.

The plurality of wings may be configured to expand radially. The lip may extend further radially than a portion of the shoulder. A proximal surface of the lip may be recessed from a proximal surface of the shoulder. The proximal surface of the lip is substantially flat. The tubular body may have an inner thread configured to engage an outer thread of the anchor insert. The tubular body may have an inner surface with at least one longitudinal channel configured to receive the repair strand. The at least one longitudinal channel may be at least partially in a common plane as the lip. The at least one longitudinal channel may include a first longitudinal channel and a second longitudinal channel opposite the first longitudinal channel. The tubular body has a plurality of barbs on an outer surface. The shoulder may have a recess configured to receive a protrusion of an outer sleeve of an inserter. The anchor sleeve may have an opening configured to receive a second repair strand. The opening may be through the lip. The lip may have an opening, wherein the second repair strand is configured to extend from the lip, loop through the tissue, and extend through the opening. The shoulder may have a circumferential channel configured to receive a second repair strand. The anchor sleeve may include a second repair strand attached to the shoulder, wherein the second repair strand is configured to secure the tissue to the shoulder. The suture anchor may include a suture threader configured to secure an end portion of a second repair strand to the shoulder. The suture threader may extend through an opening in the lip, wherein the suture threader is configured to pull the second repair strand through the opening. The lip may have a second opening, and the second repair strand is secured to the second opening. The suture threader may extend through a portion of the second repair strand and is configured to pull the end portion of the second repair strand through the portion to secure the end portion of a second repair strand to the shoulder.

A third aspect is directed to a method of securing a tissue to a bone. The method may include inserting an anchor sleeve into a bone socket of the bone; passing a first repair strand through the tissue to secure the tissue to the anchor sleeve; passing a second repair strand through the tissue; and inserting an anchor insert into the anchor sleeve to secure the second repair strand between the anchor insert and the anchor sleeve.

The method may include threading the first repair strand through an opening in the anchor sleeve. The opening is in a lip of the anchor sleeve. The method may include threading the first repair strand through a loop of a suture threader extending through the opening in the lip of the anchor sleeve, and pulling the suture threader to thread the first repair strand through the opening. The method may include threading an end portion of the first repair strand through a loop of a suture threader, and pulling the suture threader to thread the end portion of the first repair strand through a portion of the first repair strand. The first repair strand may be passed through the tissue before the anchor insert is inserted into the anchor sleeve.

A fourth aspect is directed to an anchor sleeve comprising: a tubular body configured to be inserted into a socket of a bone, wherein the tubular body has a lumen configured to receive an anchor insert, and the tubular body has a plurality of wings configured to expand; and a shoulder extending at least partially around the lumen, wherein the shoulder is configured to be seated on an outer surface of the bone, the shoulder has a raised protrusion defining a first recess and a second recess, the first recess is configured to receive a first repair strand, and the second recess is configured to receive a second repair strand.

The shoulder may have a lip, and the raised protrusion is on the lip. The lip may extend further radially than a portion of the shoulder. The lip may have a first proximal surface in the first recess and a second proximal surface in the second recess. The first proximal surface may be substantially flat, and the second proximal surface is substantially flat. The anchor sleeve may have a tubular body having a non-circular cross-section. The tubular body may have at least one flat outer surface.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In the drawings:

FIG. 1 illustrates a side view of an anchor assembly according to the present disclosure.

FIG. 2 illustrates a side view of an anchor insert and an anchor tip loaded onto a driver of the anchor assembly of FIG. 1.

FIG. 3 illustrates a side view of the driver of FIG. 2.

FIG. 4 illustrates a side view of the anchor tip of the anchor assembly of FIGS. 1-3.

FIG. 5 illustrates a perspective view of the anchor insert of the anchor assembly of FIGS. 1-4.

FIG. 6 illustrates a top view of the anchor insert of FIG. 5.

FIG. 7 illustrates a top view of an anchor sleeve of the anchor assembly of FIGS. 1-6.

FIG. 8 illustrates a perspective view of the anchor sleeve of FIG. 7.

FIG. 9 illustrates a side view of the anchor sleeve of FIGS. 7-8.

FIG. 10 illustrates an inserter for the anchor sleeve of FIGS. 7-9.

FIG. 11 illustrates a first exemplary distal tip of the inserter of FIG. 8.

FIG. 12 illustrates a second exemplary distal tip of the inserter of FIG. 8.

FIGS. 13A-C illustrate an exemplary method of anchoring a tissue to a bone with the anchor assembly of FIGS. 1-12.

FIGS. 14A-F illustrate a first exemplary method of anchoring the tissue to the bone to produce a double-row bridge repair with the anchor assembly of FIGS. 1-12.

FIG. 15A illustrates a perspective view of a second exemplary anchor sleeve of the anchor assembly of FIGS. 1-13C.

FIG. 15B illustrates a cross-sectional view of the anchor sleeve of FIG. 15A.

FIG. 15C illustrates a top view of the anchor sleeve of FIGS. 15A-B.

FIG. 15D illustrates a first side view of the anchor sleeve of FIG. 15A-C.

FIG. 15E illustrates a second side view of the anchor sleeve of FIG. 15A-D.

FIG. 15F illustrates a third side view of the anchor sleeve of FIG. 15A-E.

FIG. 16 illustrates the anchor sleeve of FIGS. 15A-F implanted into a bone.

FIG. 17 illustrates a schematic of forces applied to the anchor sleeve of FIG. 15A-D when implanted.

FIGS. 18A-B illustrate views of the anchor sleeve of FIGS. 15A-17 received on an introducer.

FIGS. 19A-I illustrate a second exemplary method of anchoring the tissue to the bone to produce a double-row bridge repair with the anchor assembly of FIGS. 15A-18B.

FIGS. 20A-D illustrate a third exemplary method of anchoring the tissue to the bone to produce a double-row bridge repair with the anchor assembly of FIGS. 15A-18B.

FIG. 21 illustrates a perspective view of a third exemplary anchor sleeve of the anchor assembly of FIGS. 1-13C.

FIG. 22 illustrates a side view of the anchor sleeve of FIG. 21.

FIG. 23 illustrates a perspective view of the anchor sleeve of FIGS. 21-22 with a repair strand and a suture threader attached.

FIG. 24 illustrates a perspective view of a fourth exemplary anchor sleeve of the anchor assembly of FIGS. 1-13C.

FIG. 25 illustrates a first side view of the anchor sleeve of FIG. 24.

FIG. 26 illustrates a second side view of the anchor sleeve of FIGS. 24-25.

FIG. 27 illustrates a perspective view of the anchor sleeve of FIGS. 24-26 with a repair strand and a suture threader attached.

FIGS. 28A-G illustrate views of a fifth exemplary anchor sleeve.

FIG. 29 illustrates a sixth exemplary anchor sleeve.

FIG. 30 illustrates a perspective view of a second exemplary anchor insert inserted in the anchor sleeve of FIG. 29.

FIG. 31 illustrates a cross-sectional view of the anchor insert inserted into the anchor sleeve of FIG. 29.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

The present disclosure provides an anchor assembly for a knotless fixation of tissue with improved fixation strength for poor bone quality and/or revision rotator cuff repair procedures. The anchor assembly may increase engagement with poor quality bone and/or improve pull-out strength. The anchor assembly may also allow expansion into larger sockets of revision or failed anchor procedures. The anchor assembly may include an anchor sleeve, an anchor tip, and an anchor insert. The anchor sleeve may be configured to be implanted into the bone socket prior to insertion of the anchor tip and the tubular member. The anchor tip may secure a repair strand, and the anchor insert may have an outer thread that matches an inner thread of the anchor sleeve. The anchor tip and/or the anchor insert may be inserted through a lumen of the anchor sleeve. The anchor sleeve may have one or more features configured to reduce rotation of the anchor sleeve relative to the bone as the anchor insert is threaded into the lumen of the anchor sleeve. For example, the anchor sleeve may have a non-circular cross-section configured to be inserted into the bone socket to reduce the rotation. The non-circular cross-section may be formed by one or more flat outer surfaces of the anchor sleeve surface. The flat outer portions may interrupt rounded outer surfaces of the anchor sleeve. As the anchor insert is inserted into the anchor sleeve, a composite thickness of the anchor insert and repair strand may expand collet style barbed wings at the distal end of the anchor sleeve. Thus, the anchor insert may have a size that does not expand the anchor sleeve unless the repair strand is present therebetween to create the composite thickness that then expands the wings of the anchor sleeve.

The anchor sleeve may have a shoulder on a proximal portion. The shoulder may provide support at the edge of the bone socket and prevent the bone socket from widening and/or the repair strand from cutting through the bone if over-tensioned. The shoulder may be shaped to provide a path for the repair strand that compresses the tissue against the bone to enhance tissue repair while minimizing potential impingement of the tissue. The shoulder may have a recess that allows the repair strand to be tension down into, protecting the repair strand and preventing the tensioned repair strand from cutting into the bone. The recess in the shoulder may further provide additional resistance to rotational forces on the anchor sleeve as the insert is rotating into the anchor sleeve. The recessed portion of the shoulder may form a lip providing an enlarged surface extending radially to provide a counter rotational force with the tensioned repair strand, reducing the tendency of the anchor sleeve from rotating laterally in the bone socket. The enlarged lip may provide further protection of the underlying bone from the tensioned repair strand to prevent the repair strand from cutting into the bone. A second repair strand may be attached to the shoulder or lip to provide additional security to the tissue repair. The second repair strand may initially secure the tissue in place before the repair strand(s) is anchored by the anchor assembly. For example, the second repair strand may be passed from the lip, through the tissue, and back to the lip. Additionally or alternatively, the second repair strand may be received through and/or around the shoulder or lip. The anchor sleeve may have teeth distal of the shoulder. For example, the teeth may be on a distal surface of the shoulder to reduce rotation of the anchor sleeve within the bone socket about a longitudinal axis, for example when the user rotates the tubular member. Thus, the anchor assembly may ensure proper positioning and orientation of the tissue along the outer surface of the bone, by reducing rotation and/or twisting of the anchor sleeve and/or repair strand during insertion and rotation of the tubular member.

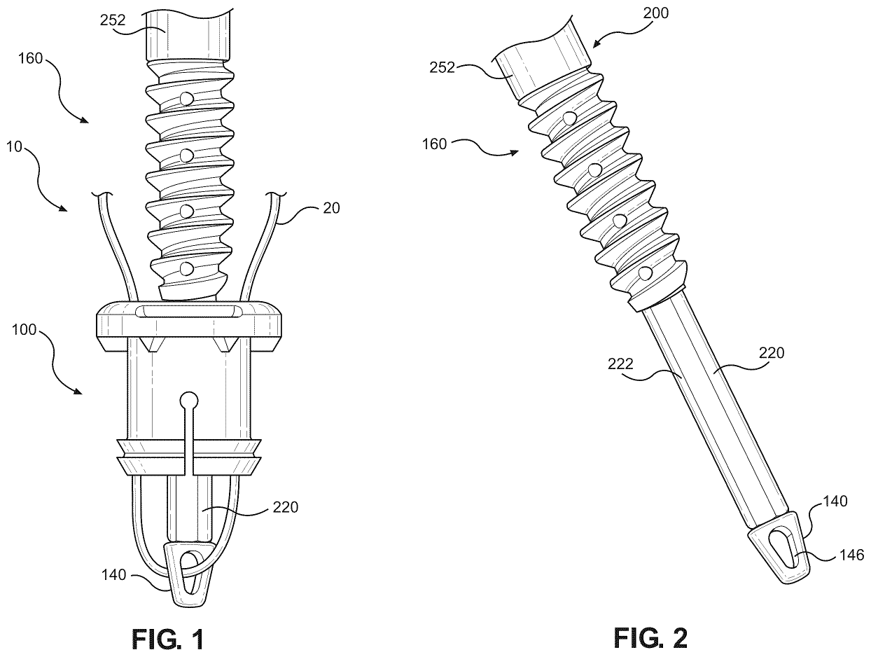

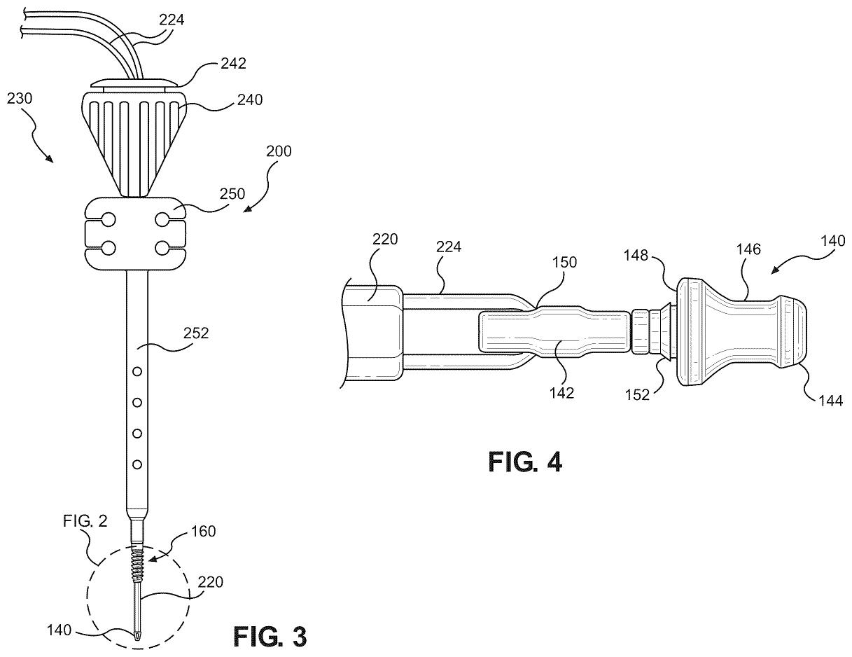

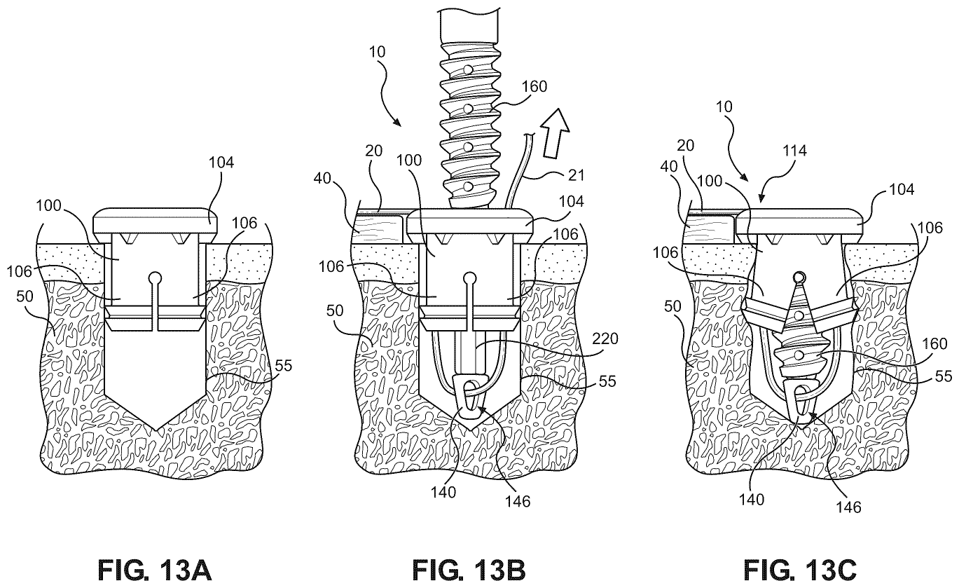

FIG. 1 illustrates an anchor assembly 10. The anchor assembly 10 may be configured to anchor a tissue 40 to a bone 50 with a repair strand 20, for example as illustrated in FIGS. 13A-C. The anchor assembly 10 may include an anchor sleeve 100 configured to be inserted into a bone socket 55 in the bone 50, an anchor tip 140 configured to receive the repair strand 20, and an anchor insert 160 configured to expand the anchor sleeve 100. As further illustrated in FIG. 2, the anchor tip 140 and the anchor insert 160 may be loaded onto a drive shaft 220 of a driver 200 for insertion into the anchor sleeve 100. As further illustrated in FIG. 3, the driver 200 may have a handle 230 including a first handle member 240 attached to the drive shaft 220 and a second handle member 250 attached to a sleeve 252 disposed around the drive shaft 220.

The anchor tip 140 may be a separate structure from the anchor sleeve 100 and the anchor insert 160. In use, the anchor insert 160 may be configured to rotate relative to the anchor tip 140 (about a longitudinal axis L of the anchor sleeve 100) to facilitate insertion of the anchor insert 160 into the anchor sleeve 100. The rotation of the anchor insert 160 relative to the anchor sleeve 100 and the anchor tip 140 may ensure proper orientation of the tissue 40 after being set and reduce strain on the repair strand 20. As illustrated in FIG. 4, the anchor tip 140 may have a proximal portion 142 and a distal portion 144. The distal portion 144 may have an opening 146 configured to receive the repair strand 20 to be secured inside of the bone socket 55. The opening 146 may have a closed periphery such as an eyelet (as shown) or have an open periphery such as a slot (as not shown). The open slot may have an opening at the distal end of the anchor tip 140 to capture the repair strand 20 as the anchor tip 140 is advanced into the bone socket 55. Alternatively, the opening of the slot may be on a lateral side to enable side loading of the repair strand 20. The distal portion 144 may have a conical shape to increase fixation strength to bone socket 55.

As further illustrated in FIG. 4, the distal portion 144 may have an outer diameter equal to or greater than an inner diameter of the drive shaft 220, such that the anchor tip 140 is configured to be retained at a distal end of the drive shaft 220, as illustrated in FIGS. 2 and 3. The opening 146 may be loaded with the repair strand 20 before being inserted into the bone socket 55. The distal portion 144 may have a proximal surface 148 configured to sit on the distal end of the drive shaft 220 (as illustrated in FIG. 2), such that the drive shaft 220 may push the anchor tip 140 into the bone socket 55.

The proximal portion 142 may be releasably secured to the distal portion of the drive shaft 220. The proximal portion 142 may have a shaft extending proximally from the distal portion 144. The proximal portion 142 may have an outer diameter smaller than the inner diameter of a lumen of the drive shaft 220, such that the proximal portion 142 may be received in the lumen of the drive shaft 220 and rotation of the drive shaft 220 does not apply torque to the anchor tip 140. The proximal portion 142 may have an aperture 150 receiving a traction suture 224 preventing the anchor tip 140 from distally separating from the drive shaft 220 during insertion. The aperture 150 may extend transversely through the proximal portion 142 such that the traction suture 224 loops through the aperture 150 from one lateral side to another lateral side. At least one (e.g., both) of the ends of the traction suture 224 may extend through the drive shaft 220 and out of a proximal opening of the driver 200 to be manipulated. The traction suture 224 may be releasably secured to the first handle member 240 to prevent inadvertent separation of anchor tip 140 from the drive shaft 220 during insertion. For example, the traction suture 224 may be circumferentially wrapped and/or looped around the first handle member 240 and/or be received in a channel 242 to secure the traction suture 224 to the handle 230. The traction suture 224 may, additionally or alternatively, be wedged and/or secured to a cleat (not shown) on the first handle member 240. After the anchor assembly 10 is implanted, the traction suture 224 may be released from the anchor tip 140 and/or the driver 200. The traction suture 224 may be subsequently used for additional tie-down of the tissue 40 after removed from the driver 200. Additionally or alternatively, the proximal portion 142 may have a protrusion 152 configured to be releasably attached to a distal end of the drive shaft 220, for example by being snapped into the lumen of the drive shaft 220.

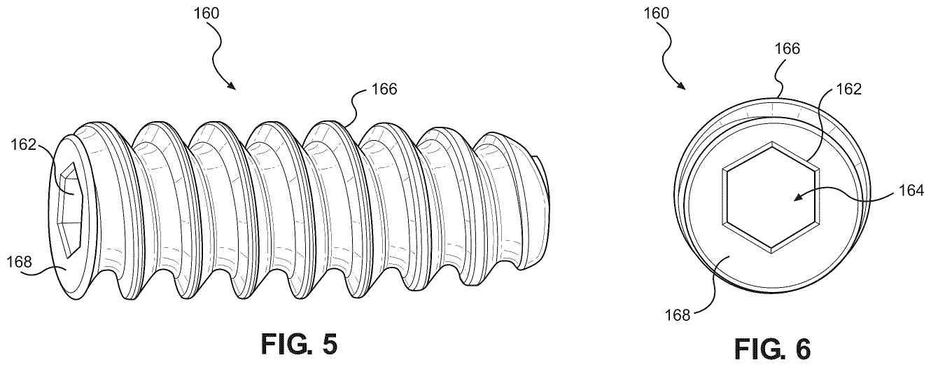

As illustrated in FIG. 2, the anchor insert 160 may be proximal of the anchor tip 140 along the drive shaft 220. As illustrated in FIGS. 5 and 6, the anchor insert 160 may have an inner surface 162 defining a lumen 164 and an outer surface defining an outer thread 166. The lumen 164 of the anchor insert 160 may have a proximal opening through a proximal surface 168 and a distal opening through a distal surface (not shown) to receive the drive shaft 220 entirely therethrough for deployment of the anchor insert 160 into the anchor sleeve 100. The inner surface 162 of the anchor insert 160 may correspond to an outer surface 222 of the drive shaft 220. A width of the inner surface 162 of the anchor insert 160 may be substantially equal to or greater than a width of an outer surface 222 of the drive shaft 220. The corresponding surfaces 162, 222 may allow the anchor insert 160 to slide relative to the drive shaft 220 during installation into the anchor sleeve 100. The corresponding surfaces 162, 222 may also allow the drive shaft 220 to apply a torque to the anchor insert 160 during installation. Each of the surfaces 162, 222 may have a non-circular cross-section to enable the drive shaft 220 to apply the torque to the anchor insert 160. As illustrated, the inner surface 162 may have a hexagonal cross-section configured to mate with a corresponding hexagonal cross-section of the outer surface 222 of the drive shaft 220. Alternatively, the corresponding cross-sections of the inner surface 162 of the anchor insert 160 and the outer surface 222 of the drive shaft 220 may be triangular, square, pentagonal, octagonal, and/or cruciform. The proximal portion 142 of the anchor tip 140 may be received in the distal opening of the lumen 164 of the anchor insert 160, such that the proximal surface 148 of the distal portion 144 may be flush against the distal surface of the anchor insert 160 when installed in the bone socket 55.

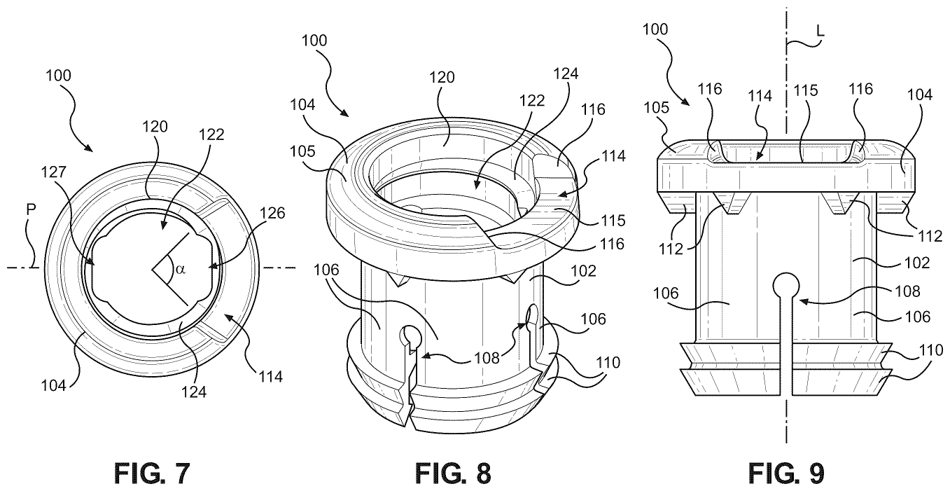

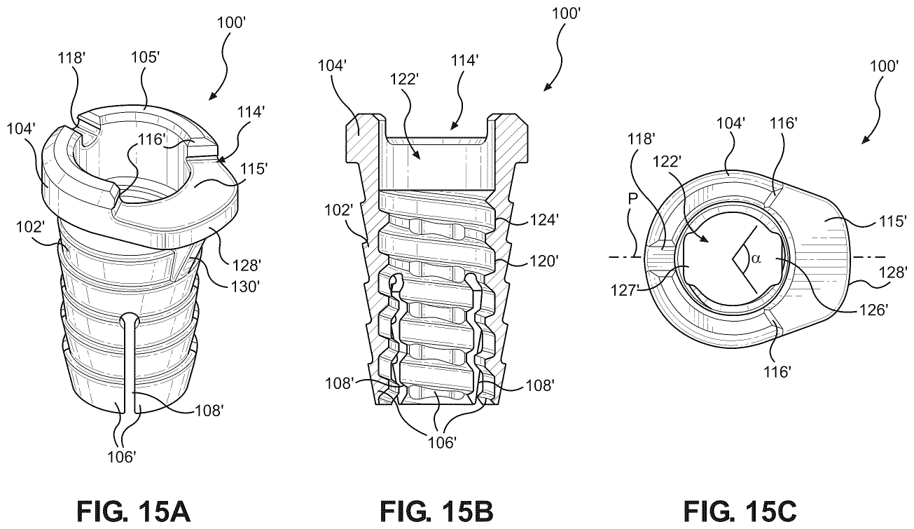

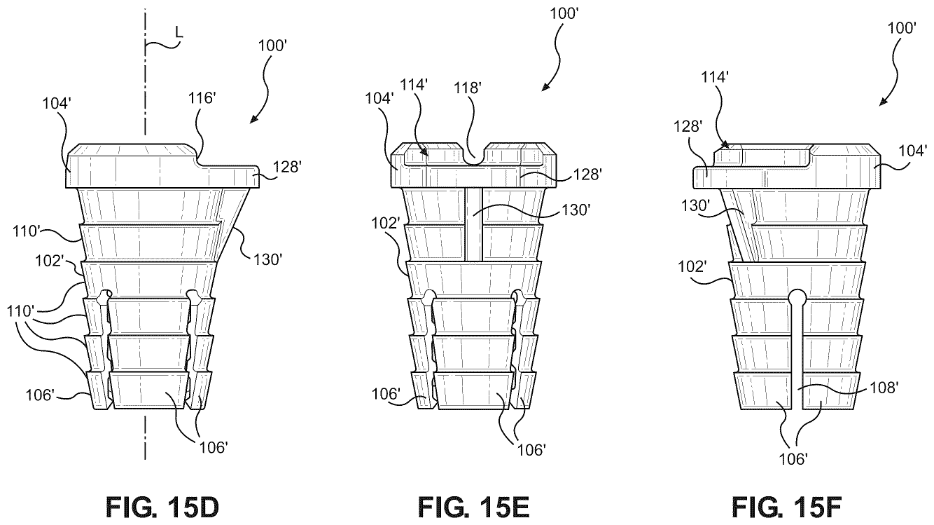

As illustrated in FIGS. 7-9, the anchor sleeve 100 may have a tubular body 102 with a shoulder 104 on a proximal portion. The tubular body 102 may have a plurality of wings 106 separated or defined by one or more longitudinal slots 108 allowing the plurality of wings 106 to expand and anchor the repair strand 20 in the bone 50 (e.g., spongy cancellous bone). As illustrated, the longitudinal slots 108 may have rounded proximal ends to reduce stress concentrations as the plurality of wings 106 expand. The one or more longitudinal slots 108 may proximally extend from the distal end of the anchor sleeve 100, such that the wings 106 have free ends forming the distal end of the anchor sleeve 100 configured to expand. The tubular body 102 may have at least one barb 110 extending circumferentially around the longitudinal axis L of the tubular body 102, for example, on the plurality of wings 106. The at least one barb 110 may be circumferentially interrupted by the one or more longitudinal slots 108. The at least one barb 110 may have a distal taper to facilitate insertion and a flat proximal surface. The flat proximal surface may form a proximal portion that is radially enlarged and/or pointed. The proximal portion of the at least one barb 110 may be configured to penetrate and/or frictionally engage the bone when expanded to increase pull-out strength. The at least one barb 110 may include a plurality of barbs 110 spaced longitudinally apart along the plurality of wings 106. The at least one barb 110 may include two or more barbs 110 on a distal portion of the tubular body 102, as illustrated in FIGS. 8 and 9. Additionally or alternatively, the plurality of barbs 110 may extend substantially the entire length of the tubular body 102, as illustrated in FIGS. 15A-C, 21-27. The shoulder 104 may remain substantially fixed as the plurality of wings 106 expand.

The plurality of wings 106 may be configured to expand radially. For example, the plurality of wings 106 may be configured to pivot outwardly along a radius with respect to the longitudinal axis L from a fixed proximal portion of the tubular body 102. The pivoting of the plurality of wings 106 may be due to the interference fit of the anchor insert 160 and the separation at the longitudinal slots 108. Additionally or alternatively, the plurality of wings 106 may be configured to expand tangentially. For example, the insertion of the anchor insert 160 may cause the plurality of wings 106 to expand in a direction tangential of a circumference of the tubular body 102.

As illustrated in FIG. 9, the anchor sleeve 100 at the shoulder 104 may have a width or diameter greater than at least a portion of the tubular body 102. The shoulder 104 may be a flange configured to engage or be seated on an outer surface of the bone (e.g., hard, cortical bone) when the tubular body 102 is received in the bone socket 55. The shoulder 104 may extend at least partially around the longitudinal axis L of the anchor sleeve 100. The shoulder 104 may extend completely around the longitudinal axis L of the anchor sleeve 100, as illustrated in FIGS. 7-9. Alternatively, the shoulder 104 may extend less than the entirety of the circumference of the anchor sleeve 100 (not shown).

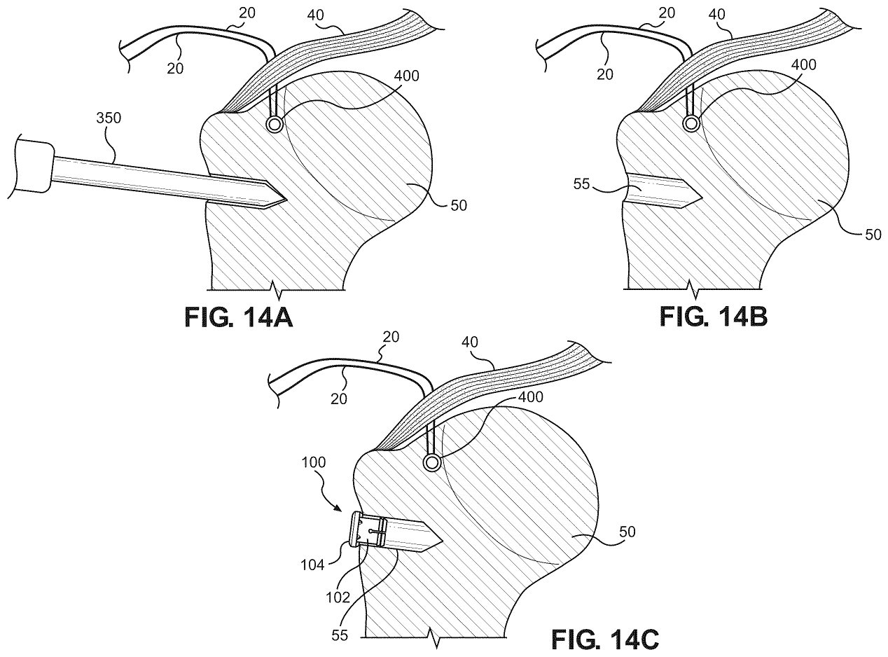

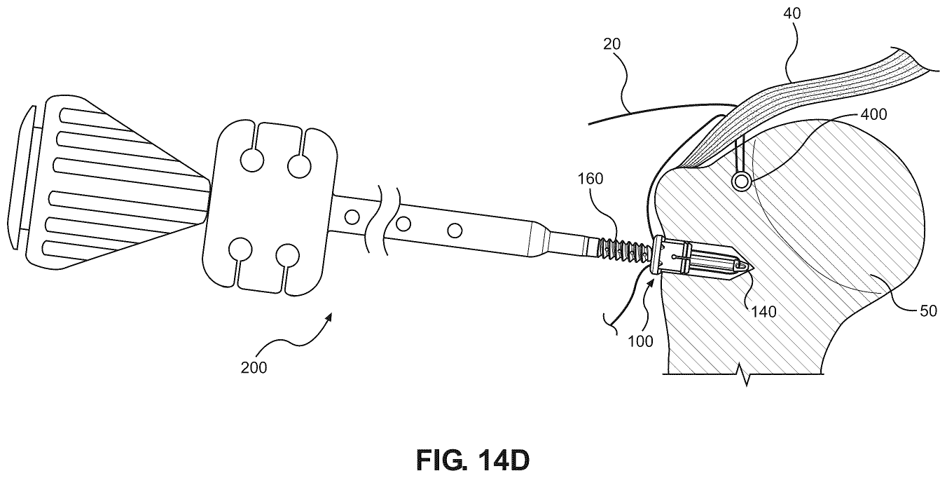

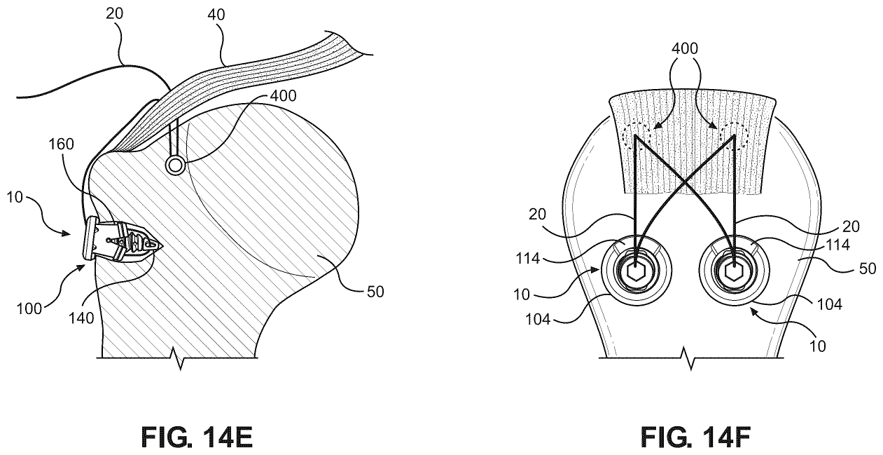

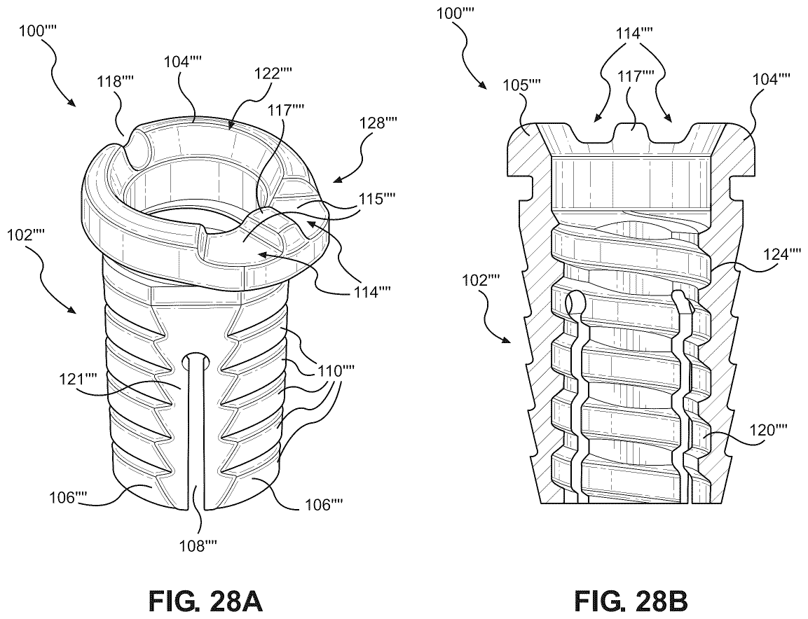

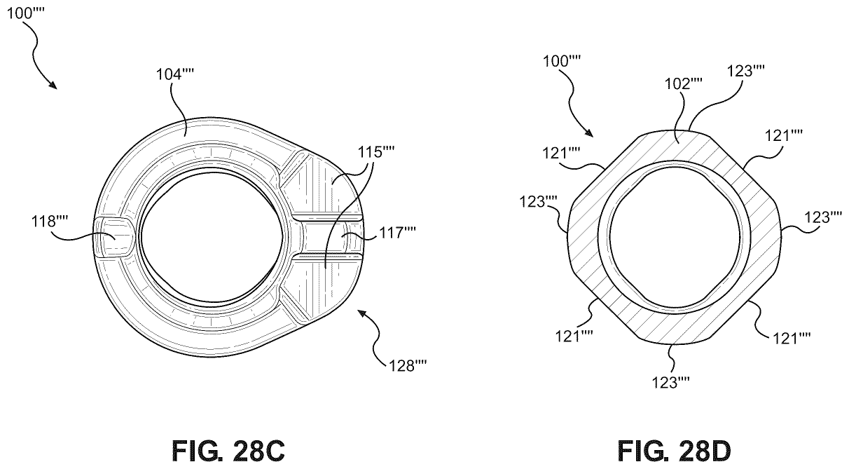

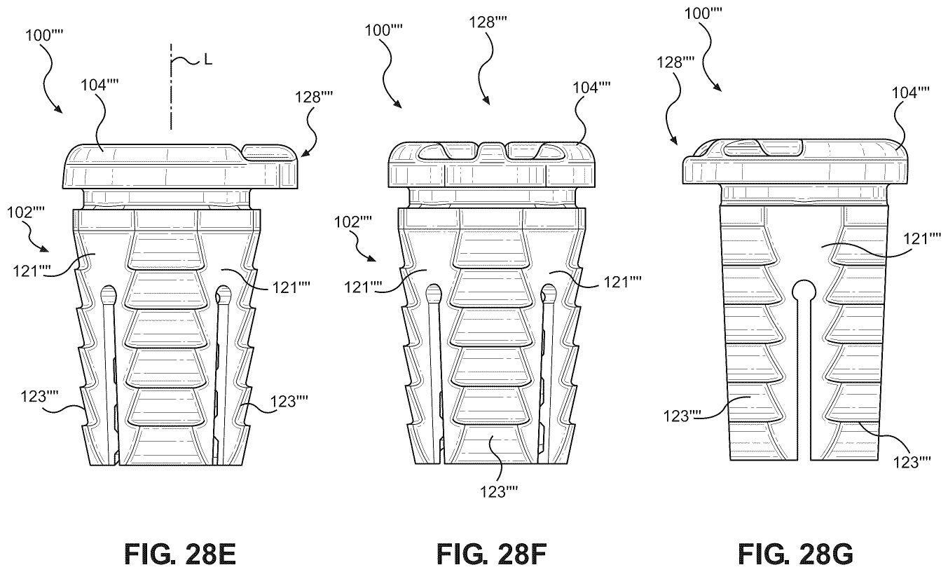

The shoulder 104 may provide support at the edge of the bone socket for the repair strand 20 and prevent the repair strand 20 from widening the socket or cutting through bone if over-tensioned. The shoulder 104 may have a proximal surface 105 that is rounded on at least a portion of (e.g., a majority of) the proximal portion to reduce stress on any overlying tissues. The repair strand 20 may be tensioned down onto the shoulder 104 to secure the tissue 40 to the bone 50. The proximal surface 105 of the shoulder 104 may have at least one recess 114, each configured to receive a repair strand 20 when tensioned down. Each recess 114 may have a surface 115 forming a seat for the repair strand 20, as illustrated in FIGS. 13B-C. The surface 115 may be substantially flat, as further illustrated. Additionally or alternatively, the surface 115 may have at least a concave portion (as illustrated in FIGS. 22, 26) configured to receive the repair strand 20. Additionally or alternatively, the shoulder 104 may have a raised partition forming a first recess and a second recesses (as illustrated in FIGS. 28A-G) each configured to receive a repair strand 20. The at least one recess 114 may protect the repair strand 20 when implanted to prevent wear and tear when implanted. The recess 114 may optimize the path of the repair strand 20 by positioning the repair strand 20 closer to the bone 50, as further illustrated in FIGS. 13B-C. The path of the repair strand 20 may ensure full contact of the tissue 40 with the bone 50 and increase the footprint of the compression, allowing for accelerated healing of the tissue 40 to the bone 50. The recess 114 may further provide rotational resistance reducing the tendency of the anchor sleeve 100 to rotate. The tension of the repair strand 20 may reduce rotation of the anchor sleeve 100 about the longitudinal axis L by a compressive force against the bone 50 and/or frictionally engaging the surface 115 of the recess 114. For example, the repair strand 20 may be a flat tape having an increased surface to frictionally engage the anchor sleeve 100 at the surface 115. The repair strand 20 may, additionally or alternatively, reduce rotation of the anchor sleeve 100 about the longitudinal axis L by engaging a vertical wall 116 on at least one lateral side of the recess 114. As illustrated in FIG. 7, the recess 114 of the shoulder 104 may extend at an angle α of at least about 10° around the longitudinal axis L of the anchor sleeve 100. The angle α may be about 10° to about 180° around the longitudinal axis L of the anchor sleeve 100. For example, the angle α of the recess 114 may be about 80° to about 130° around the longitudinal axis L of the anchor sleeve 100, such as about 90° to about 120°. The angle α of the recess 114 may substantially match and/or exceed a width and/or intended separation of one or more repair strands 20 to minimize rotation of the anchor sleeve 100. The angle α of the recess 114 may be sufficiently wide enough to receive repair strands 20 from spaced apart medial anchors 400 in a double-row bridge repair, as illustrated in FIG. 14F. The angle α may be greater than 180°. One of the vertical walls 116 may be contacted by the repair strand 20 and provide a stop to reduce further rotation of the anchor sleeve 100 when the insert 160 is rotationally threaded into the anchor sleeve 100.

A plurality of teeth 112 may be distal of the shoulder 104 to secure and/or increase rotational resistance of the anchor sleeve 100. The plurality of teeth 112 may engage the bone before insertion of the anchor insert 160 and reduce rotation of the anchor sleeve 100 as the anchor insert 160 is threaded into the lumen 122. The plurality of teeth 112 may extend from the shoulder 104 and along the tubular body 102. For example, the plurality of teeth 112 may extend distally from a distal surface of the shoulder 104 and radially from the tubular body 102 to engage the outer surface of the bone as the shoulder 104 is seated. The plurality of teeth 112 may be configured to penetrate and/or frictionally engage the outer surface of the bone. The plurality of teeth 112 may be large and spaced apart about 15° to about 180° along the distal surface of the shoulder 104 around the longitudinal axis L of the anchor sleeve 100. For example, as illustrated, the shoulder 104 may have about four to twelve (e.g., six) teeth 112 evenly spaced apart around the longitudinal axis L of the anchor sleeve 100.

As further illustrated in FIGS. 7 and 8, the anchor sleeve 100 may have an inner surface 120 defining a lumen 122 configured to receive the anchor insert 160. The inner surface 120 may have an inner thread 124 configured to engage the outer thread 166 of anchor insert 160 through rotation. The inner thread 124 may be preformed on the inner surface 120. Additionally or alternatively, the inner thread 124 may be formed by barbs or protrusions spaced apart on the inner surface 120. Additionally or alternatively, the inner surface 120 may be a soft surface such that the inner thread 124 is formed during insertion of the anchor insert 160. The anchor insert 160 may be sized to substantially match the lumen 122 of the anchor sleeve 100, such that the anchor insert 160 may be inserted and be threadedly secured in the lumen 122 of the anchor sleeve 100 in the absence of the repair strand 20 without expanding the anchor sleeve 100. An outer width or diameter of the anchor insert 160 may be substantially equal to or less than an inner width or diameter of the anchor sleeve 100. A major width or diameter of the thread 166 may be substantially equal to or less than a width or major diameter of the thread 124, such as the portion of the inner surface 120 without the thread 124. A minor width or diameter of the thread 166 may be substantially equal to or less than a minor width or diameter of the thread 124, such as the inner width or diameter of the thread 124. Thus, a composite thickness of the anchor insert 160 and the repair strand 20 may be required to expand the anchor sleeve 100.

The inner thread 124 may be discontinuous along the circumference of the inner surface 120. The inner thread 124 may be interrupted by at least one longitudinal channel 126, 127 extending along and in communication with the lumen 122, forming two separated threaded portions on the inner surface 210. The at least one longitudinal channel 126, 127 may provide a recess configured to receive the repair strand 20 when the anchor insert 160 is received in the lumen 122 of the anchor sleeve 100. The at least one longitudinal channel 126, 127 may have a first longitudinal channel 126 on a first side of the lumen 122 and a second longitudinal channel 127 on a second side of the lumen 122. The first longitudinal channel 126 and the second longitudinal channel 127 may be offset by about 180° around the lumen 122 to receive the repair strand 20 on opposite sides of the anchor insert 160. The first channel 126 and/or the second channel 127 may be at least partially in a common plane P as the recess 114, as illustrated in FIG. 7. The common plane P may be a central plane extending through the longitudinal axis L of the anchor sleeve 100. As illustrated in FIGS. 13B and 13C, the repair strand 20 may extend laterally from the side of the tissue 40, through the recess 114 of the shoulder 104, distally through the first longitudinal channel 126 along the anchor insert 160, laterally through the opening 146 of the anchor tip 140, and proximally through the second longitudinal channel 127 along the anchor insert 160.

The anchor sleeve 100, the anchor tip 140, and/or the anchor insert 160 may be formed of a biocompatible and/or biosorbable material, such as a polymer, metal, and/or biological material. The anchor sleeve 100, the anchor tip 140, and/or the anchor insert 160 may be formed of a bioabsorbable material, such as poly-(L-lactic acid) (PLLA), poly-(D,L-lactide), and poly glycolic acid (PGA), for example, or other bioabsorbable, non-metallic materials, which may be especially tailored for hardness, tensile strength and compressive strength. Additionally or alternatively, the anchor sleeve 100, the anchor tip 140, and/or the anchor insert 160 may be formed of a metal such as titanium, titanium alloy, stainless steel or stainless steel alloy. Other biocompatible materials which could be used include plastics, allograft bone and/or inert bone substitute materials.

The repair strand 20 may be a high-strength suture, formed from at least one fiber of ultra-high molecular weight polyethylene (UHMWPE). The repair strand 20 may be a FiberWire® suture, produced by Arthrex and disclosed in U.S. Pat. No. 6,716,234, the entire disclosure of which is incorporated herein by reference. FiberWire® suture may be formed of at least one UHMWPE fiber braided with at least one other fiber to form lengths of suture material. The FiberWire® suture may include a core within a hollow braided construct, the core being a twisted yarn of UHMWPE. Additionally or alternatively, the repair strand 20 may include TigerWire® suture, FiberChain® suture, or TightRope® suture (all produced by Arthrex), although other cored or coreless sutures may be utilized. Additionally or alternatively, the repair strand 20 may be a flat suture tape, such as FiberTape® produced by Arthrex. Additionally or alternatively, the repair strand 20 may be an allograft and/or have a biological component. The allograft and/or biological component may be tendon or pericardium to provide improved tissue repair. Any combination of suture, suture tape, an allograft, and/or biological component may be employed, depending on the characteristics of the specific surgical repair and/or as desired.

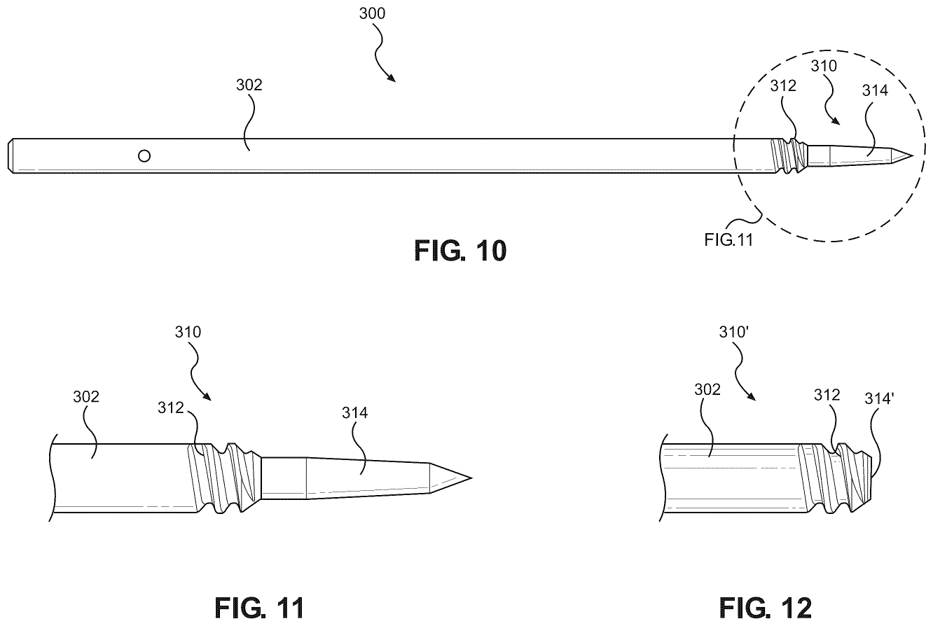

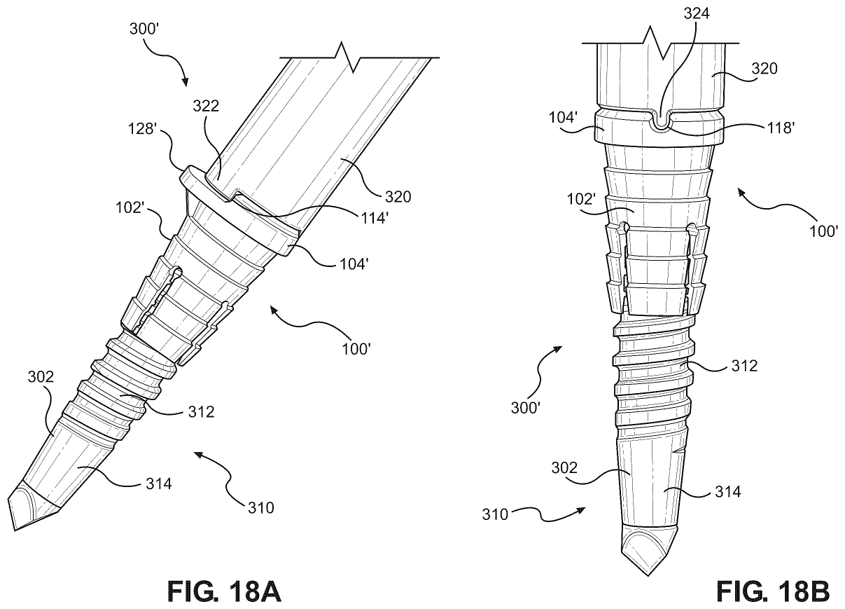

FIGS. 10-12 illustrate a sleeve inserter 300 configured to insert the anchor sleeve 100 into the bone 50. The sleeve inserter 300 may have a shaft 302 configured to be attached to a manual or motorized handle (not shown) actuated by a user. The shaft 302 may be longitudinally and rotationally fixed to the handle to allow the user to advance and/or rotate the shaft 302 to insert the anchor sleeve 100 into the bone 50. For example, similar to the drive shaft 220, the shaft 302 may have a non-circular cross-section corresponding to a non-circular cross-section of the handle, such that the handle may apply a torque to the shaft 302. Additionally or alternatively, the handle and/or shaft 302 may have a collet configured to rotationally secure the shaft 302 to the handle. The shaft 302 may have a distal tip 310 to releasably engage the anchor sleeve 100. As further illustrated, the distal tip 310 may have an outer thread 312 configured to releasably engage the inner thread 124 of the anchor sleeve 100. The outer thread 312 may be configured such that rotation of the shaft 302 in a first direction may insert the distal tip 310 into the lumen 122 to releasably secure the anchor sleeve 100 onto the distal tip 310 through engagement of the inner thread 124. The distal tip 310 may then be engaged to the bone 50 and push the anchor sleeve 100 into the bone socket 55. The shaft 302 may then be rotated in a second direction opposite of the first direction to disengage the outer thread 312 from the inner thread 124 and retract the distal tip 310 from the lumen 122 of the anchor sleeve 100. The sleeve inserter 300 may have an outer sleeve 320 (e.g., as illustrated in FIGS. 18A-B) that may provide a longitudinal and/or rotational counterforce by pushing the anchor sleeve 100 to secure the anchor sleeve 100 in the bone socket 55 as the sleeve inserter 300 is retracted. For example, as illustrated in FIGS. 18A-B and incorporated herein by reference, the outer sleeve 320 may have one or more distal protrusions 322, 324 configured to positively interlock with one or more recesses 114, 118′ to reduce relative rotation between the anchor sleeve 100 and the outer sleeve 320.

Furthermore, the teeth 112 of the anchor sleeve 100 may reduce relative rotation and/or secure the anchor sleeve 100 to the bone 50 as the shaft 302 is rotationally removed from the lumen 122. As illustrated in FIGS. 10 and 11, the distal tip 310 may be self-punching and be configured to insert the anchor sleeve 100 without any need of pre-forming a hole in the bone 50. As illustrated in FIG. 12, the sleeve inserter 300 have a distal tip 310′ with a distal end 314′ that is flat, blunt and/or rounded and be used with a separate instrument (e.g., a punch 350, FIG. 14A) that pre-forms the socket 55 in the bone 50.

Returning to FIG. 3, the first handle member 240 of the handle 230 may be attached to the drive shaft 220 and the second handle member 250 of the handle 230 may be attached to the sleeve 252 disposed around the drive shaft 220. The first handle member 240 may be proximal of the second handle member 250. The handle 230 may be manipulated to position the anchor tip 140 and the anchor insert 160 into the anchor sleeve 100. The anchor tip 140 may be held at the distal end of the drive shaft 220, and the anchor insert 160 may be received around the drive shaft 220 on the outer surface 222. Through manipulation of the handle 230, the drive shaft 220 may initially push the anchor tip 140 through the lumen of the anchor sleeve 100 and into the bone socket 55 and position the anchor insert 160 at the proximal opening of the anchor sleeve 100. The drive shaft 220 may be rotated to advance the anchor insert 160 through the lumen 164, for example by rotating the first handle member 240 relative to the second handle member 250. The rotation of the first handle member 240 may apply torque to the drive shaft 220 to cause rotation of the anchor insert 160 through engagement of the surfaces 162, 222. The second handle member 250 may be held stationary with one hand as the first handle member 240 is rotated with another hand. The engagement of the threads 124, 166 may cause advancement of the anchor insert 160 through the lumen 122 of the anchor sleeve 100. Further discussion of the anchor tip 140, the anchor insert 160, and the driver 200 is provided in U.S. Pat. No. 10,820,897, the entire disclosure of which is incorporated herein by reference.

FIGS. 13A-C illustrate a method of using the anchor assembly 10. The method may be used to secure a tissue 40 to a bone 50. The tissue 40 may be a soft tissue or bone. For example, the method may be for repairing a torn rotator cuff by attaching the tissue 40 to its original site on the bone 50 and in its original orientation. Thus, the tissue 40 may be a supraspinatus tendon, and the bone 50 may be a humerus of a human shoulder. However, the method may have a variety of other applications in securing tissue 40 to bone 50. For example, the tissue 40 may be a bone such as a talus, and the bone 50 may be a fibula in performing AC joint stabilization.

As illustrated in FIG. 13A, a bone socket 55 may be formed into the bone 50. The bone socket 55 may be formed by the sleeve inserter 300 with the self-punching distal tip 310, as illustrated in FIG. 11. Alternatively, the bone socket 55 may be pre-formed by a different instrument (e.g., a punch 350, FIG. 14A) prior to inserting the sleeve inserter 300. The anchor sleeve 100 may be inserted with the sleeve inserter 300 into the bone sockets 55. The sleeve inserter 300 may push the tubular body 102 of anchor sleeve 100 into the bone socket 55 and engage the outer surface of the bone 50 with the shoulder 104. The teeth 112 may secure and/or reduce rotation of the anchor sleeve 100 about the longitudinal axis L relative to the bone 50 as the outer thread 312 of the inserter 300 is rotated out of engagement of the inner thread 124 of the anchor sleeve 100 as the inserter 300 is removed. Additionally or alternatively, the outer sleeve (e.g., 320, FIG. 18A-B) of the sleeve insert 300 positioned on the shaft 302 may provide a counterforce to reduce rotation and/or longitudinal translation of the anchor sleeve 100 and prevent withdrawal. For example, as illustrated in FIGS. 18A-B and incorporated herein for sake of brevity, the outer sleeve 320 may have one or more distal protrusions 322, 324 configured to positively interlock with one or more recesses 114, 118′ to reduce relative rotation between the anchor sleeve 100 and the outer sleeve 320. Additionally or alternatively, a non-circular cross-section of the anchor sleeve 100 (as illustrated in FIGS. 28A-G) may reduce rotation of the anchor sleeve 100 when inserted.

As illustrated in FIG. 13B, the repair strand 20 may be secured to the tissue 40. For example, the repair strand 20 may be looped through the tissue 40 with a suture passer, such as that described in U.S. Pat. No. 7,972,344 as incorporated herein by reference in its entirety. After the repair strand 20 is passed through the tissue, the repair strand 20 may be fed through the opening 146 of the anchor tip 140 with a suture threader (not shown). The repair strand 20 may be looped through the tissue 40, such that two free ends of the repair strand 20 may be passed through the opening 146 of the anchor tip 140. Alternatively, the repair strand 20 may be looped through the tissue 40 with a slip knot, so a single free end may be passed through the opening 146. The tissue 40 may be positioned on the outer surface of the bone 50 in the desired position and orientation for repair.

The anchor tip 140 may be inserted with the driver 200 through the anchor sleeve 100 into the bone socket 55, with the drive shaft 220 pushing the anchor tip 140 and the traction suture 224 holding the anchor tip 140 against the drive shaft 220. The driver 200 may position the anchor tip 140 at the bottom of the bone socket 55 or otherwise at a desired depth. The repair strand 20 may be configured such that at least one length of the repair strand 20 extends from the tissue 40 through the recess 114 of the shoulder 104, through the anchor sleeve 100 along the first longitudinal channel 126, through the opening 146 of the anchor tip 140, and back through the anchor sleeve 100 along the second longitudinal recess 127.

The repair strand 20 may be tensioned by pulling at least one length 21 of the repair strand 20 extending from the second longitudinal channel 127, as illustrated in FIG. 13B. The tensioning of the repair strand 20 may secure the tissue 40 directly against the bone 50, enhancing the footprint of compression and accelerating healing of the tissue 40. The thickness of the shoulder 104 may accommodate the thickness of the tissue 40, minimizing potential impingement of the repair strand 20 against the tissue 40. The shoulder 104 may also prevent the repair strand 20, when tensioned, from cutting into the bone 50. The driver 200 may position the distal end of the anchor insert 160 outside of the bone socket 55 at or near the proximal opening of the anchor sleeve 100 with the anchor tip 140 positioned in the bone socket 55.

As illustrated in FIG. 13C, the anchor insert 160 may be advanced into the bone socket 55 and through the lumen 122 of the anchor sleeve 100. The anchor insert 160 may be advanced by rotating the drive shaft 220. The outer thread 166 of the anchor insert 160 may engage the inner thread 124 of the anchor sleeve 100 to advance the anchor insert 160 through the anchor sleeve 100. The anchor insert 160 may advance along the drive shaft 220 toward the anchor tip 140, as the drive shaft 220 rotates the anchor insert 160. A composite thickness of the anchor insert 160 and the repair strand 20 may expand the anchor sleeve 100, such that the anchor insert 160 and the repair strand 20 may apply a compressive force against the longitudinal channels 126, 127 to expand the plurality of wings 106. The at least barb 110 may penetrate into the spongy cancellous bone. Tension applied to the tissue 40 by the repair strand 20 may be evaluated. The tension on the repair strand 20 may be adjusted by backing out the anchor insert 160 with the driver 200 and reentry of the anchor insert 160. After the desired tension is achieved by the anchor assembly 10, the traction suture 224 may be released. The drive shaft 220 may be detached from the anchor tip 140 and proximally withdrawn from the anchor insert 160 and the anchor sleeve 100. The at least one length 21 of the repair strand 20 extending from the anchor sleeve 100 may be trimmed after final placement of the anchor assembly 10. Additional bone sockets 55 may be formed, and additional anchor assemblies 10 may be inserted into the additional bone sockets 55. The method may be performed in a single-or a double-row bridge repair of the rotator cuff.

FIGS. 14A-F illustrate a first method of using the anchor assembly 10 in attaching a tissue 40 to a bone 50 in a double-row bridge repair. For example, the method may be for repairing a torn rotator cuff by attaching the tissue 40 back to its original site on the bone 50 and in its original orientation. Thus, the tissue 40 may be a supraspinatus tendon, and the bone 50 may be a humerus of a human shoulder. Alternatively, the tissue 40 may be an Achilles tendon and the bone may be a heel bone such as a calcaneus. However, the method of FIGS. 14A-F may have applications in securing other types of tissue 40 to other types of bone 50. The entire disclosure of FIGS. 13A-C is incorporated herein for sake of brevity.

As illustrated in FIG. 14A, one or more repair strands 20 may be anchored to the bone 50 with one or more medial anchors 400. For example, the one or more medial anchors 400 may include a plurality of medial anchors 400 anchored to the bone 50 in a row to form a medial row. The repair strand 20 may be anchored into the bone 50 with the medial anchor 400. The medial anchor(s) 400 may be implanted such that each repair strand 20 has a pair of ends extending from the bone 50, as illustrated in FIG. 14A-F. The ends of the repair strand 20 may be the same repair strand 20 or different repair strands 20. The ends of the repair strands 20 may be passed through the tissue 40 by being looped through the tissue 40 with a suture passer, such as that described in U.S. Pat. No. 7,972,344 as incorporated herein by reference in its entirety, and discussed with reference to FIG. 13A-B. The one or more medial anchors 400 may have a number of different structural designs, including those as discussed herein. Additionally or alternatively, the one or more medial anchors 400 may be as described in U.S. Pat. No. 10,820,897, as incorporated herein by reference in its entirety. Additionally or alternatively, the one or more medial anchors 400 may be a soft anchor, as described in U.S. Pat. No. 10,441,408 as incorporated herein by reference in its entirety. As represented by the suture construct of FIG. 14F, two medial anchors 400 may be inserted into the bone 50, with each medial anchor 400 having two ends of a repair strand 20 extending therefrom. However, additional medial anchors 400 may be used.

As further illustrated in FIG. 14A-C, a punch 350 may be used to form one or more bone sockets 55. The anchor sleeve 100 may be inserted into one or more of the bone sockets 55. For example, the punch 350 may have a distal end that is pointed and/or sharpened to form the bone socket 55. The quality of the bone 50 may be determined based on the insertion of the punch 350 and/or the resultant bone socket 55 formed by the punch 350. Alternatively, the quality of the bone 50 may be determined before insertion of the punch 350 according to clinical, visual, and/or tactile indications. Based on a determination of the quality of the bone being poor, the anchor sleeve 100 may be inserted into each pre-formed bone socket 55 as illustrated in FIG. 14C. The anchor sleeve 100 may be inserted with the sleeve inserter 300 having the distal tip 310′, as illustrated in FIG. 12. Alternatively, the one or more bone sockets 55 may be formed by the sleeve inserter 300 with the self-punching distal tip 310 as the anchor sleeve 100 is inserted, to perform the operations of FIGS. 14A-C in a single motion. The one or more bone sockets 55 and/or the one or more anchor sleeves 100 may be lateral of the one or more medial anchors 400. The one or more bone sockets 55 may include a plurality of bone sockets 55 and/or the one or more anchor sleeves 100 may include a plurality of anchor sleeves 100, formed in a row lateral of the medial row of the medial anchors 400. The one or more anchor sleeves 100 may be inserted into the one or more of the bone sockets 55 such that the shoulder 104 may engage or be seated on an outer surface of the bone 50 and the tubular body 102 may be received in the bone socket 55. Furthermore, the shoulder 104 may be oriented such that the recess 114 is in the direction of or aligned with the tissue 40, thus being at least partially in a common plane P to form a direct suture path from the lumen 122 to the tissue 40, as illustrated in FIGS. 7, 13B, C and 14F.

As illustrated in FIG. 14D, the one or more repair strands 20 may be threaded through the anchor tip 140, and the anchor tip 140 may be inserted through the anchor sleeve 100 and into the bone 50 by pushing with the driver 200. An end of the repair strand 20 extending from each medial anchor 400 may be threaded through each anchor tip 140, for example to form the suture construct FIG. 14F. As illustrated in FIG. 14D, a first end of the repair strand 20 may be threaded through the anchor tip 140 of a first anchor assembly 10, before the other end of the repair strand 20 is threaded to a second anchor tip 140 of a second anchor assembly 10.

As illustrated in FIG. 14E, the driver 200 may then insert the anchor insert 160 into the anchor sleeve 100. The driver 200 may rotate the anchor insert 160 to threadedly advance the anchor insert 160 through the lumen 122 of the anchor sleeve 100. The anchor insert 160 may expand the anchor sleeve 100 with the composite thickness of the anchor insert 160 and the repair strand 20. The expansion of the anchor sleeve 100 may be through radial deflection of the plurality of wings 106. The driver 200 may then be removed from the anchor tip 140 and the anchor insert 160. The repair strand 20 may be trimmed on the lateral side with respect to the medial anchors 400 once the desired tensioned is achieved and the repair strand 20 is secured in the anchor assembly 10, as illustrated in FIGS. 13B-C and 14E. The steps of FIGS. 14D-E may be repeated with a second anchor assembly 10 to secure second ends of the repair strands 20.

As illustrated in FIG. 14F, a double-row bridge anchoring construct may be formed. The anchoring construct may be quick and formed with no knots and only two suture-passing steps. The anchoring construct may also provide a broad footprint that can be helpful for repairs to degenerative cuff tissue for which tissue pull-through may be a concern. The ends of the repair strands 20 may provide a bridge repair securing the tissue 40 to the bone 50 by forming a bridge between the medial anchor 400 and the anchor assembly 10. The anchor sleeve 100 may provide improved fixation strength for poor bone quality and/or revision procedures. The recess 114 of the shoulder 104 for each anchor sleeve 100 may have a width providing a suture path for a pair of repair strands 20 extending from at least two medial anchors 400. The recess 114 may reduce wear on the repair strand 20 and/or bone 50 and/or reduce rotation of the anchor sleeve 100 about the longitudinal axis L. Further discussion of exemplary methods is described in U.S. Pat. No. 8,419,794, as incorporated herein by reference in its entirety.

The anchor sleeve 100 may be used in one or more (e.g. all) of the bone sockets 55. In some methods, the anchor sleeve 100 may be inserted in less than all of the bone sockets 55 of the method. For example, the anchor sleeve 100 may be only used in bone socket(s) 55 having reduced bone quality and/or requiring revised anchor procedures. Thus, the method may include additional steps of inserting at least one additional anchor assembly 10 with and/or without the anchor sleeve 100. For example, the method may include inserting a first anchor assembly 10 into a first bone socket 55 by inserting the anchor insert 160 into the anchor sleeve 100 (as illustrated in FIGS. 13A-C) and inserting a second anchor assembly 10 into a second bone socket 55 by inserting the anchor insert 160 into the bone socket 55 without the anchor sleeve 100, such that the anchor insert 160 is directly engaged and/or threaded to the bone (as discussed in U.S. Pat. No. 8,419,794 and incorporated herein by reference in its entirety). The second anchor assembly 10 (without an anchor sleeve 100) may be rotated and/or pushed into the respective bone socket 55. Accordingly, a kit may include a first anchor assembly 10 with a first anchor sleeve 100 (as described herein) and a second anchor assembly 10 with a second anchor sleeve 100 (as described herein), where the first anchor assembly 10 and the second anchor assembly 10 may be substantially identical. Additionally or alternatively, the kit may include a third anchor assembly 10 without an anchor sleeve 100. The third anchor assembly 10 may be configured to be rotated (as discussed herein) and/or pushed into the respective bone socket 55 to directly engage the bone 50.

FIGS. 15A-F illustrate a second exemplary anchor sleeve 100′ for an anchor assembly 10′. The anchor sleeve 100′ may have one or more of the same features as discussed with reference to the anchor sleeve 100, the entire disclosure of which is incorporated herein for sake of brevity. For example, the anchor sleeve 100′ may have a tubular body 102′ with a shoulder 104′ on a proximal portion. The tubular body 102′ may have a plurality of wings 106′ separated or defined by one or more longitudinal slots 108′ allowing the plurality of wings 106′ to expand and anchor the repair strand 20 in the bone 50 (e.g., spongy cancellous bone). The tubular body 102′ may have at least one barb 110′ extending circumferentially around the longitudinal axis L of the tubular body 102′. The at least one barb 110′ may have a plurality of barbs 110′. The at least one barb 110′ may be formed on the plurality of wings 106′. The at least one barb 110′ on the plurality of wings 106′ may be circumferentially interrupted by the one or more longitudinal slots 108′. Additionally or alternatively, the at least one barb 110′ may be proximal of the plurality of wings 106′ and be uninterrupted around the circumference. As illustrated, the at least one barb 110′ may extend substantially the entire length of the tubular body 102′. The at least one barb 110′ may have a distal taper to facilitate insertion and a flat proximal surface. The shoulder 104′ may remain substantially fixed as the plurality of wings 106′ expand.

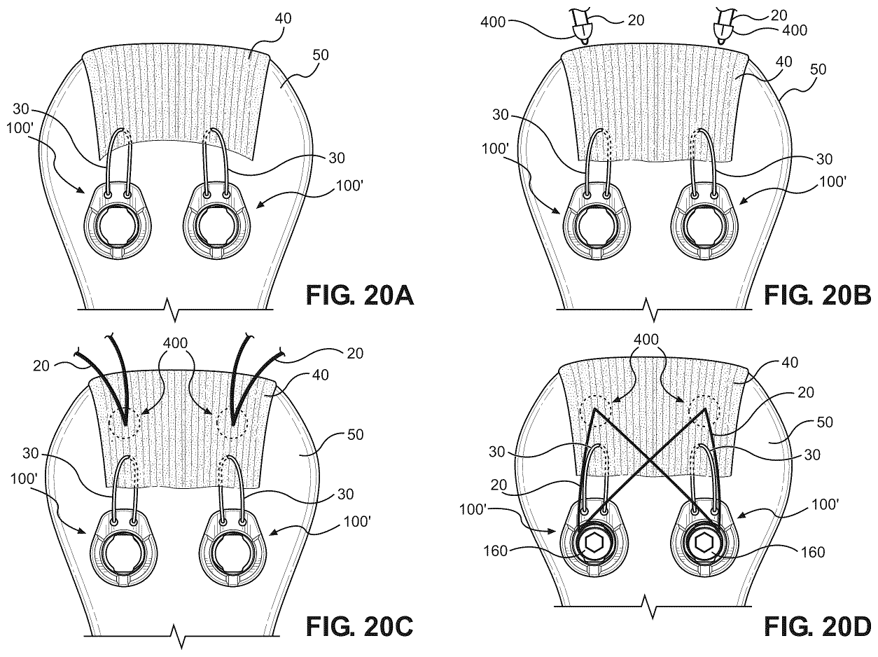

As illustrated, the anchor sleeve 100′ at the shoulder 104′ may have a width or diameter greater than at least a portion of the tubular body 102′. The shoulder 104′ may be configured to engage or be seated on an outer surface of the bone (e.g., hard, cortical bone) when the tubular body 102′ is received in the bone socket 55. The shoulder 104′ may extend at least partially around the longitudinal axis L of the anchor sleeve 100′. The shoulder 104′ may extend completely around the longitudinal axis L of the anchor sleeve 100′, as illustrated in FIGS. 7-9. Alternatively, the shoulder 104′ may extend less than the entirety of the circumference of the anchor sleeve 100′ (not shown). The shoulder 104′ may provide support at the edge of the bone socket 55 for the repair strand 20 and prevent the repair strand 20 from widening the socket or cutting through bone if over-tensioned. The shoulder 104′ may have a proximal surface 105′ that is rounded on at least a portion of (e.g., a majority of) the proximal portion to reduce stress on any overlying tissues. The repair strand 20 may be tensioned down onto the shoulder 104′ to secure the tissue 40 to the bone 50. The proximal surface 105′ of the shoulder 104′ may have a recess 114′ configured to receive the repair strand 20 when tensioned down. The recess 114′ may have a surface 115′ forming a seat for the repair strand 20. The surface 115′ may be substantially flat, as illustrated. The recess 114′ may protect the repair strand 20 when implanted to prevent wear and tear when implanted. The recess 114′ may optimize the path of the repair strand 20 by positioning the repair strand 20 closer to the bone 50. The path of the repair strand 20 may ensure full contact of the tissue 40 with the bone 50 and increase the footprint of the compression, allowing for accelerated healing of the tissue 40 to the bone 50. The recess 114′ may further provide rotational resistance reducing the tendency of the anchor sleeve 100′ to rotate. The tension of the repair strand 20 may reduce rotation of the anchor sleeve 100′ about the longitudinal axis L by frictionally engage the surface 115′ of the recess 114′. For example, the repair strand 20 may be a flat tape having an increased surface to frictionally engage the anchor sleeve 100 at the surface 115′. The repair strand 20 may, additionally or alternatively, reduce rotation of the anchor sleeve 100 about the longitudinal axis L by engaging vertical walls 116′ on lateral sides of the recess 114′. Similar to the first exemplary recess 114, the recess 114′ of the shoulder 140′ may extend at an angle α of at least about 10° around the longitudinal axis L of the anchor sleeve 100′. The angle α may be about 10° to about 180° around the longitudinal axis L of the anchor sleeve 100′, as illustrated in FIG. 15C. For example, the angle α of the recess 114 may be about 80° to about 130° around the longitudinal axis L of the anchor sleeve 100′, such as about 90° to about 120°. The angle α of the recess 114 may substantially match and/or exceed a width and/or intended separation of one or more repair strands 20 to minimize rotation of the anchor sleeve 100. The angle α of the recess 114′ may be sufficiently wide enough to receive repair strands 20 from spaced apart medial anchors 400 in a double-row bridge repair, as illustrated in FIG. 20D. However, the angle α of the recess 114′ may be sufficiently narrow to reduce rotation of the anchor sleeve 100. A plurality of teeth may extend distally from a distal surface of the shoulder 104′, as discussed with reference to FIGS. 8 and 9.

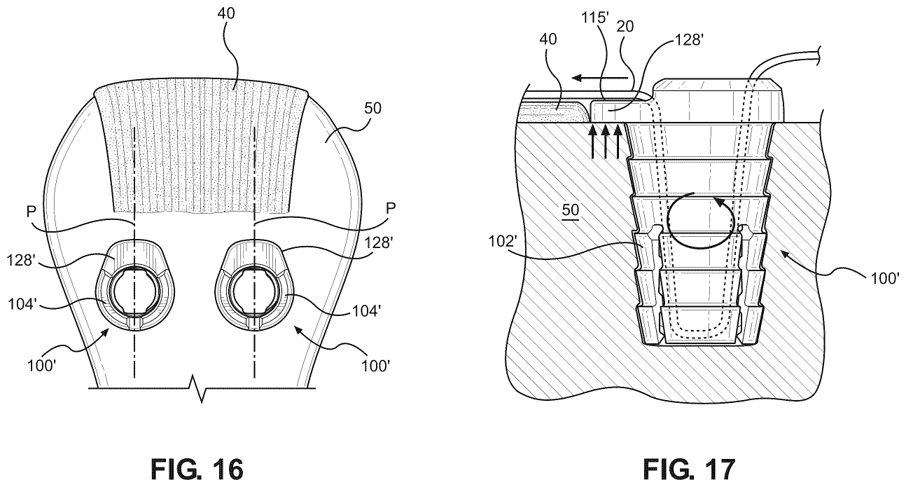

The shoulder 104′ may form a lip 128′ extending at least partially around the longitudinal axis L. The lip 128′ may be recessed from the proximal surface 105′ by the recess 114′ and define the surface 115′ configured to seat the repair strand 20. As illustrated in FIG. 16, the anchor sleeve 100′ may be inserted into the bone 50 to align the recess 114′ and the lip 128′ with the tissue 40 to provide a favorable suture path, as discussed with reference to FIGS. 13B-C. The lip 128′ may face the medial side of the tissue 40 to keep the one or more repair strands 20 in the recess 114′ of the anchor sleeve 100′ to take advantage of the side walls 116′ to help reduce rotation of the anchor sleeve 100′.

The lip 128′ may extend further radially than a portion of the shoulder 104′ to improve the load distribution of the repair strand 20 onto the bone 50, as further illustrated in FIG. 17. The lip 128′ may extend further radially than a majority of the shoulder 104′. For example, the lip 128′ may extend further radially than the non-recessed portion of the shoulder 104′ that is not intended to support the repair strand 20. The lip 128′ many have an increased surface around to provide leverage to counteract rotation of the anchor sleeve 100′. The lip 128′ may improve the pull-out strength as the lip 128′ prevents the anchor sleeve 100′ from rotating out of the bone socket 55. As the repair strand 20 is pulled in the direction of the tissue 40, the repair strand 20 will tend to rotate the anchor sleeve 100′ about a lateral axis with respect to the bone socket 55. However, the lip 128′ may provide a vertical force against the outer surface of the bone 50 to counteract the torque and resist the rotation about the lateral axis improving the security. For example, the lip 128′ may reduce rotational movement of the tubular body 102′ that would cause widening of the bone socket 55 and loosening of the anchor sleeve 100′. The lip 128′ may be reinforced by a rib 130′ extending between the tubular body 102′ and the rib 130′. The rib 130′ may reinforce the lip 128′ rotationally with respect to the tubular body 102′ to reduce deformation and/or breakage of the lip 128′.

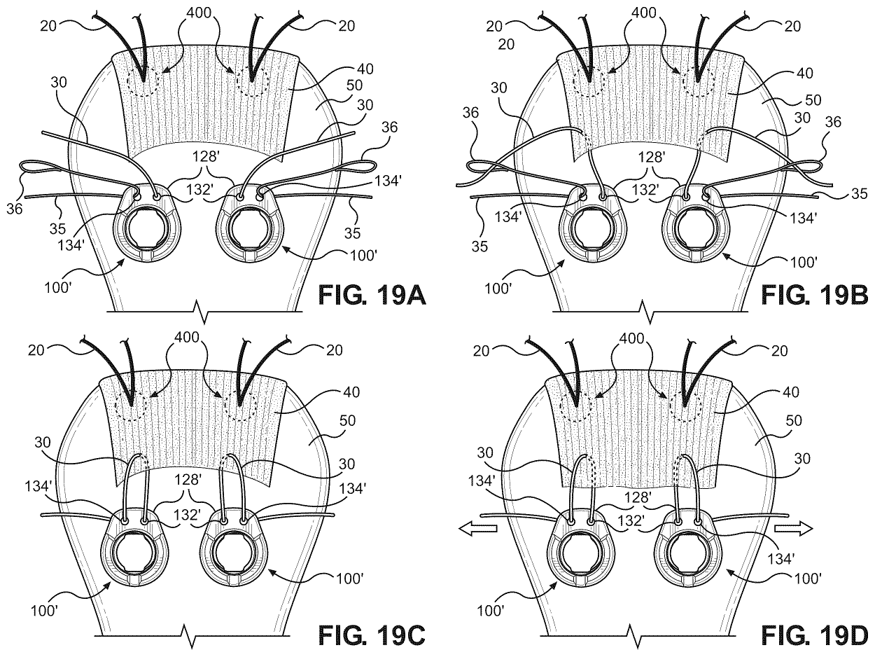

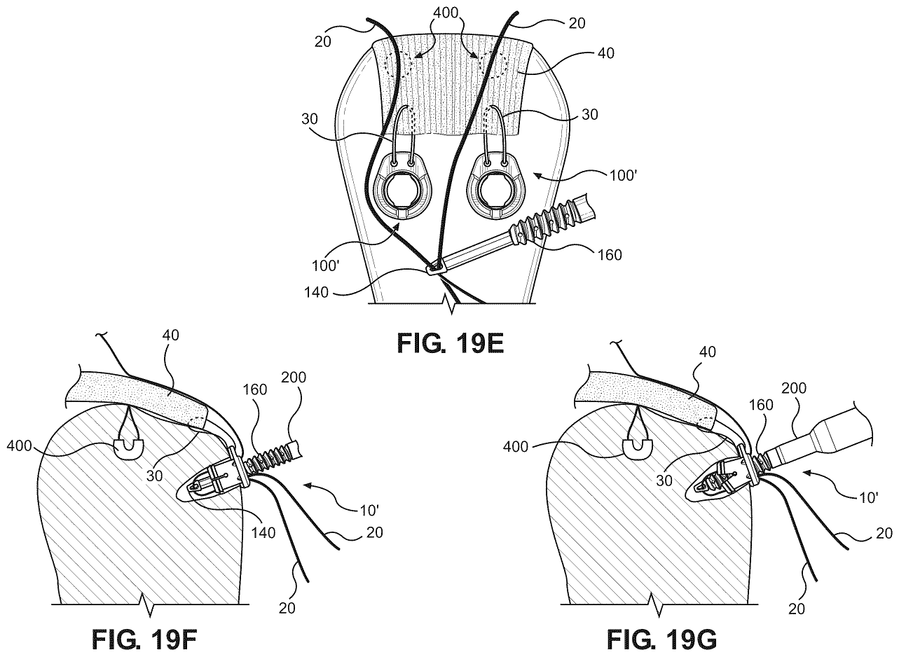

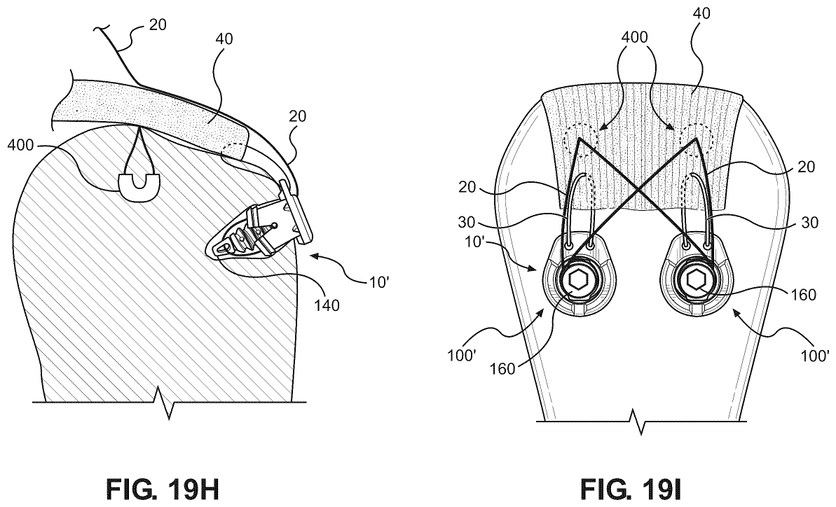

The lip 128′ may have one or more holes 132′, 134′ extending through its thickness, as illustrated in FIGS. 19A-E. A second repair strand 30 may be secured or fixed to the shoulder 104′ at the lip 128′ by being passed through a first hole 132′. The second repair strand 30 may have a first end portion that is spliced, knotted, welded, or otherwise secured through the first hole 132′ to prevent the second repair strand 30 from being pulled out of the first hole 132′. For example, the second repair strand 30 may be knotted to provide a diameter larger than the first hole 132′ to prevent the end of the second repair strand 30 from being pulled therethrough. However, the second repair strand 30 may be secured or fixed to the lip 128′ without the first hole 132′, such as being welded or integrally formed with the lip 128′. A second end portion of the second repair strand 30 may be free to be looped through the tissue 40 to secure the tissue 40. A suture threader 35 may be secured to the lip 128′ by being passed through the second hole 134′. The suture threader 35 may be threaded through the second hole 134′ such that a first free end may be manipulated or pulled by the user and a second free end portion may have a loop 36 that may receive the second repair strand 30. In use, the free end portion of the second repair strand 30 may be passed through the tissue 40 and be threaded through the loop 36 in the suture threader 35. The suture threader 35 may then be pulled to thread the free end portion of the second repair strand 30 through the second hole 134′ to secure the tissue 40 to the lip 128′. Thus, the second repair strand 30 may extend from the lip 128′ of the anchor sleeve 100′, through the tissue 40, and back to the lip 128′ of the anchor sleeve 100′. The second repair strand 30 may temporarily secure the tissue 40 to the lip 128′. Additionally or alternatively, the second repair strand 30 may permanently secure the tissue 40, for example by knotting, welding, or otherwise securing the second repair strand 30 to the second hole 134′ and cutting the free end portion of the second repair strand 30. The second repair strand 30 may allow the tissue 40 to be reduced and then a more robust fixation may be produced from the repair strand 20 that bridges the medial anchor 400 and the anchor assembly 10′. The second repair strand 30 may embody the same structures and/or materials as that described with respect to the repair strand 20, as incorporated herein for sake of brevity. The shoulder 104′ may have a second recess 118′ configured to receive a protrusion of an outer sleeve 320. The second recess 118′ may be narrower than the recess 114′ because the second recess 118′ is not intended to receive a repair strand 20. The second recess 118′ may be opposite of the recess 114′, for example, offset by about 180°around the longitudinal axis L and in the common plane P. The recesses 114′, 118′ may provide a positive, interlocking engagement with the outer sleeve 320.

As illustrated in FIGS. 15B-C, the anchor sleeve 100′ may have an inner surface 120′ defining a lumen 122′ configured to receive the anchor insert 160. The inner surface 120′ may have an inner thread 124′ configured to engage the outer thread 166 of anchor insert 160 through rotation. The anchor insert 160 may be sized to substantially match the lumen 122′ of the anchor sleeve 100′, such that the anchor insert 160 may be inserted and be threadedly secured in the lumen 122′ of the anchor sleeve 100′ in the absence of the repair strand 20 without expanding the anchor sleeve 100′. An outer width or diameter of the anchor insert 160 may be substantially equal to or less than an inner width or diameter of the anchor sleeve 100′. A major width or diameter of the thread 166 of the anchor insert 160 may be substantially equal to or less than a width or major diameter of the thread 124′ of the anchor sleeve 100′, such as the portion of the inner surface 120′ without the thread 124′. A minor width or diameter of the thread 166 may be substantially equal to or less than a minor width or diameter of the thread 124′, such as the inner width or diameter of the thread 124′. Thus, a composite thickness of the anchor insert 160 and the repair strand 20 may be required to expand the anchor sleeve 100′.

The inner thread 124′ may be discontinuous along the circumference of the inner surface 120′. The inner thread 124′ may be interrupted by at least one longitudinal channel 126′, 127′ extending along and in communication with the lumen 122. The at least one longitudinal channel 126′, 127′ may provide a recess configured to receive the repair strand 20 when the anchor insert 160 is received in the lumen 122′ of the anchor sleeve 100′. The at least one longitudinal channel 126′, 127′ may have a first longitudinal channel 126′ on a first side of the lumen 122′ and a second longitudinal channel 127′ on a second side of the lumen 122′. The first longitudinal channel 126′ and the second longitudinal channel 127′ may be offset by about 180° around the lumen 122′ to receive the repair strand 20 on opposite sides of the anchor insert 160. The first channel 126′ and/or the second channel 127′ may be at least partially in a common plane P as the recess 114′ and/or the lip 128′, as illustrated in FIGS. 15C, 16. The common plane P may be a central plane extending through the longitudinal axis L of the anchor sleeve 100′. The repair strand 20 may extend laterally from the side of the tissue 40, through the recess 114′ of the shoulder 104, distally through the first longitudinal channel 126′ along the anchor insert 160, laterally through the opening 146 of the anchor tip 140, and proximally through the second longitudinal channel 127′ along the anchor insert 160.

FIGS. 18A-B illustrate a sleeve inserter 300′ configured to insert the anchor sleeve 100′ into the bone 50. The sleeve inserter 300′ may have one or more of the same features as discussed with reference to the sleeve inserter 300, the entire disclosure of which is incorporated herein for sake of brevity. For example, the sleeve inserter 300′ may have a shaft 302 configured to be attached to a manual or motorized handle (not shown) actuated by a user. The shaft 302 may have a distal tip 310 to releasably engage the anchor sleeve 100′. As further illustrated, the distal tip 310 may have an outer thread 312 configured to releasably engage the inner thread 124′ of the anchor sleeve 100′. The outer thread 312 may be configured such that rotation of the shaft 302 in a first direction may insert the distal tip 310 into the lumen 122′ to releasably secure the anchor sleeve 100′ onto the distal tip 310 through engagement of the inner thread 124′. The distal tip 310 may then be engaged to the bone 50 and push the anchor sleeve 100 into the bone socket 55. The shaft 302 may then be rotated in a second direction opposite of the first direction to disengage the outer thread 312 from the inner thread 124′ and retract the distal tip 310 from the lumen 122′ of the anchor sleeve 100′. The sleeve inserter 300 may have an outer sleeve 320 that may provide counterforce to the anchor sleeve 100′ during removal of the shaft 302 from the bone socket 55. The outer sleeve 320 may provide a longitudinal force to retain the anchor sleeve 100′ in the bone socket 55 by pushing the anchor sleeve 100′ as the shaft 302 is retracted. Additionally, or alternatively, the outer sleeve 320 may provide a rotational force allowing the shaft 302 to be rotated relative to the anchor sleeve 100′. The outer sleeve 320 may have a first distal protrusion 322 configured to be received in the recess 114′ of the anchor sleeve 100′ and a second distal protrusion 324 configured to be received in the recess 118′. The protrusions 322, 324 may provide a positive, interlocking engagement between the outer sleeve 320 and the anchor sleeve 100′ to reduce relative rotation, allowing the shaft 302 to be rotated relative to the anchor sleeve 100′. The 180°offset of the recess 114′, 118′ may improve the engagement to counteract torque applied by the shaft 302. As illustrated in FIGS. 18A-B, the distal tip 310 may be self-punching and be configured to insert the anchor sleeve 100′ without any need of pre-forming a hole in the bone 50. As illustrated in FIG. 12, the sleeve inserter 300 have a distal tip 310′ with a distal end 314′ that is flat, blunt and/or rounded and be used with a separate instrument (e.g., a punch 350, FIG. 14A) that pre-forms the socket 55 in the bone 50.

FIGS. 19A-I illustrate an exemplary method of anchoring the tissue 40 to the bone 50 to produce a double-row bridge repair. The method may include one or more steps as illustrated and discussed with reference to FIGS. 13A-C and 14A-F, the entire disclosure of which is incorporated herein for sake of brevity. The one or more medial anchors 400 may be inserted into the bone 50, as discussed and illustrated with reference to FIG. 14A. The repair strand 20 from each of the medial anchors 400 may have first and second ends extending through the tissue 40. The anchor sleeve 100′ may be inserted, as illustrated and discussed with reference to FIGS. 14B-C, 16.

As illustrated in FIG. 19A, each of the anchor sleeves 100′ may be inserted to align the lip 128′ with the tissue 40 along the plane P (FIG. 16). The second repair strand 30 may be secured or fixed to the first hole 132′ and the suture threader 35 may be threaded through the second hole 134′. As illustrated in FIG. 19B, the free end portion of the second repair strand 30 for each of the anchor sleeves 100′ may be passed through the tissue 40 and be threaded through the loop 36 at the second end portion of the suture threader 35. As illustrated in FIG. 19C, the first end portion of the suture threader 35 for each of the anchor sleeves 100′ may be pulled to thread the free end portion of the second repair strand 30 through the second hole 134′ and remove the suture threader 35 from the second hole 134′. Thus, the second repair strand 30 may extend from the lip 128′ of the anchor sleeve 100′, through the tissue 40, and back to the lip 128′ of the anchor sleeve 100′. As illustrated in FIG. 19D, the free end portion of the second repair strand 30 for each of the anchor sleeves 100′ may be further pulled or tensioned to manipulate or position the tissue 40 in a desired position. The free end portion of the second repair strand 30 may then be secured or fixed to the lip 128′ with a knot and/or weld, and an excess length of the free end portion of the second repair strand 30 may be cut.

As illustrated in FIG. 19E, a repair strand 20 from each of two medial anchors 400 may be threaded through the opening 146 of the anchor tip 140 after the one or more repair strands 30 initially reduce and position the tissue 40 by being secured to the anchor sleeve 100′. As illustrated in FIG. 19F, the anchor tip 140 may then be inserted into the anchor sleeve 100′ and the bone socket 55 by pushing with the driver 200. As illustrated in FIG. 19G, the anchor insert 160 may be inserted into the anchor sleeve 100′ to expand the anchor sleeve 100′ by being rotating with the driver 200, as discussed for example with reference to FIGS. 13A-C. As illustrated in FIG. 19H, the driver 200 may be removed, and an excess length of the repair strand 20 may be cut. The steps of FIGS. 19E-G may be repeated with a second anchor assembly 10 to second ends of the repair strands 20.

As illustrated in FIG. 19I, a double-row bridge anchoring construct may be formed. The second repair strands 30 may allow the tissue 40 to be reduced before the repair strand 20 is secured and/or anchored with the anchor assembly 10′, thus a more robust fixation may be produced by the one or more repair strands 20 that bridge the medial anchor 400 and the anchor assembly 10′. Additionally or alternatively, the repair strands 20, 30 may provide two or more independent, permanent anchoring mechanisms to reinforce the bridge repair, providing a more robust fixation of the tissue 40 to the bone 50.

FIGS. 20A-D illustrate an exemplary method of anchoring the tissue to the bone to produce a double-row bridge repair. The method of FIGS. 20A-D may be similar to that described with reference to FIGS. 19A-I, the entire disclosure of which is incorporated herein for sake of brevity. For example, as illustrated in FIG. 20A, the anchor sleeve 100′ may be inserted and secured to the tissue 40 with the second repair strand 30, as discussed with reference to FIGS. 19A-D. However, as further illustrated in FIGS. 20A-B, the anchor sleeves 100′ may inserted into the bone 50 to reduce or position the tissue 40 before inserting the medial anchors 400. As illustrated in FIG. 20B-C, once the tissue 40 is reduced, the medial anchors 400 may be inserted directly through the tissue 40. The repair strand 20 of the medical anchors 400 may then be fixated into anchor sleeve 100′ with the anchor tip 140 and the anchor insert 160, for example as discussed with reference to FIGS. 19E-G. The at least one second repair strand 30 may facilitate the method of FIGS. 20A-D by securing the tissue 40 independently of the medial anchor 400 and/or repair strand 20.

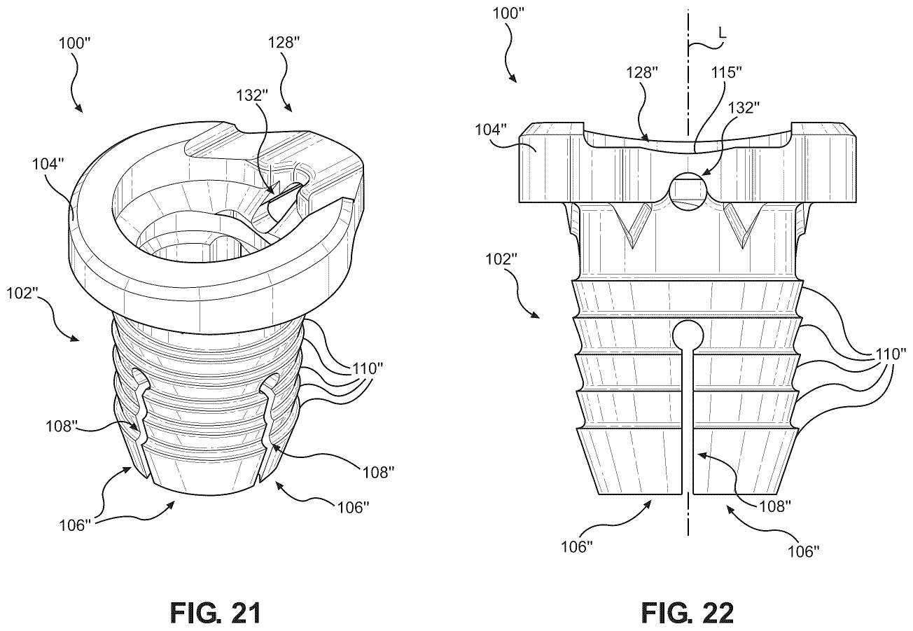

FIGS. 21-23 illustrate a third exemplary anchor sleeve 100″ for an anchor assembly. The anchor sleeve 100″ may have one or more of the same features as discussed with reference to the anchor sleeve 100 and/or 100′, the entire disclosures of which is incorporated herein for sake of brevity. Furthermore, the anchor sleeve 100″ may be used in the same methods and systems as described herein, the entire disclosures of which is incorporated herein for sake of brevity.

As previously discussed, the anchor sleeve 100″ may have a tubular body 102″ with a shoulder 104″ on a proximal portion. The tubular body 102″ may have a plurality of wings 106″ separated or defined by one or more longitudinal slots 108″ allowing the plurality of wings 106″ to expand and anchor the repair strand 20 in the bone 50 (e.g., spongy cancellous bone). The tubular body 102″ may have at least one barb 110″ extending circumferentially around the longitudinal axis L of the tubular body 102″. The at least one barb 110″ may have a plurality of barbs 110″. The at least one barb 110″ may be formed on the plurality of wings 106″. The at least one barb 110″ on the plurality of wings 106″ may be circumferentially interrupted by the one or more longitudinal slots 108″. Additionally or alternatively, the at least one barb 110″ may be proximal of the plurality of wings 106″ and be uninterrupted around the circumference. As illustrated, the at least one barb 110″ may extend substantially the entire length of the tubular body 102″. The at least one barb 110″ may have a distal taper to facilitate insertion and a flat proximal surface. The shoulder 104″ may remain substantially fixed as the plurality of wings 106″ expand.

The shoulder 104″ may form a lip 128″ extending at least partially around the longitudinal axis L. The anchor sleeve 100″ may have a hole and/or channel 132″, for example extending through the lip 128″ and/or the tubular body 102″. The hole 132″ may extend through a junction of the lip 128″ and the tubular body 102″, as illustrated in FIGS. 21-22.

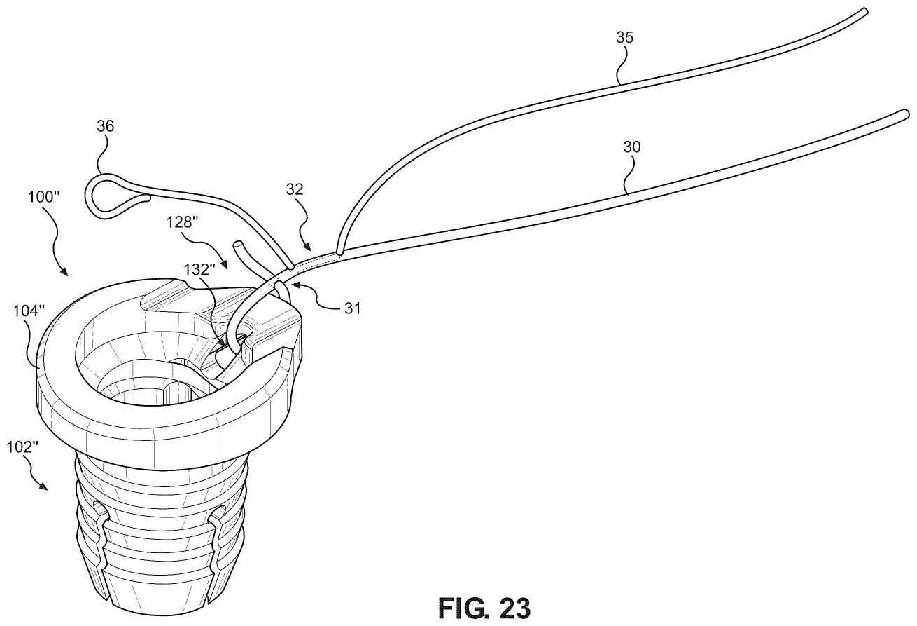

As illustrated in FIG. 23, a second repair strand 30 may be secured or fixed to the shoulder 104″ at the lip 128″ by being passed through the hole 132″. The second repair strand 30 may secure the tissue 40 to the bone 50 as similarly discussed with reference to FIGS. 19A-20D, the entire disclosure of which is incorporated herein for sake of brevity. The second repair strand 30 may have a first end portion that is spliced, knotted, welded, or otherwise secured to the hole 132″ to prevent the second repair strand 30 from being pulled out of the hole 132″. For example, as illustrated, the second repair strand 30 may be passed or looped through the hole 132″ and extend through itself to secure the first end portion of the second repair strand 30 to the anchor sleeve 100″. The second repair strand 30 may be spliced through itself at a first portion 31, such that the second repair strand 30 extends perpendicular of a longitudinal axis of the second repair strand 30. The lip 128″ may have a concave upper surface 115″ proximal of or above the hole 132″ (as illustrated in FIG. 22) configured to receive the repair strand 20 and/or the second repair strand 30. A second end portion of the second repair strand 30 may be free to be looped through the tissue 40 to secure the tissue 40. A suture threader 35 may be secured to the second repair strand 30 by extending through a longitudinal length of the second repair strand 30, for example by being spliced through a second portion 32 of the second repair strand 30. The suture threader 35 may extend from the second repair strand 30 such that a first free end portion may be manipulated or pulled by the user and a second free end portion may have a loop 36 that may receive the second repair strand 30. In use, the free end portion of the second repair strand 30 may be passed through the tissue 40 and threaded through the loop 36 in the suture threader 35. The suture threader 35 may then be pulled proximally from the second portion 32 to thread the free end portion of the second repair strand 30 through the second portion 32 as the loop 36 of the suture threader 35 is pulled through and proximally out of the second portion 32. Thus, a first end portion of the second repair strand 30 may extend through the hole 132″ of the anchor sleeve 100″ and be secured, for example by extending through itself at a first portion 31. A second end portion of the second repair strand 30 may be looped through the tissue 40 and secured to itself by extending through the second portion 32.

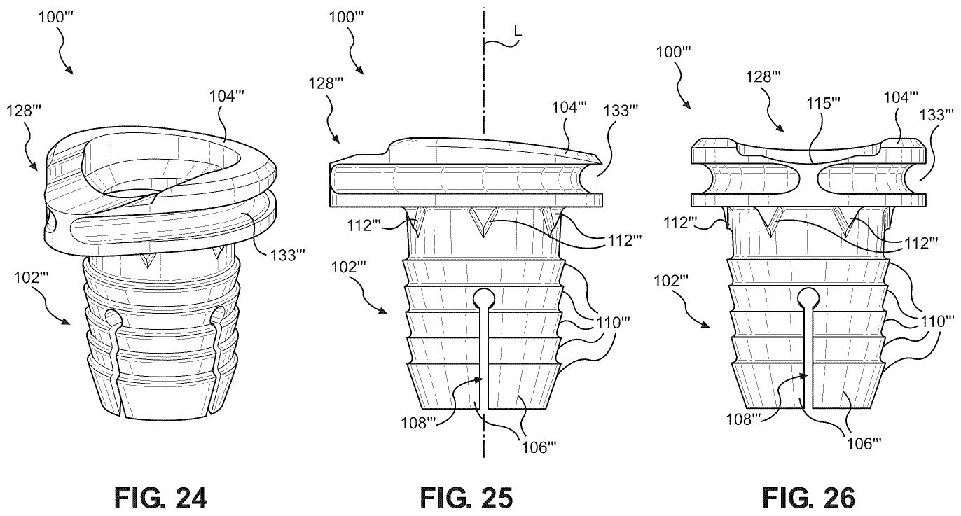

FIGS. 24-27 illustrate a fourth exemplary anchor sleeve 100″′ for an anchor assembly. The anchor sleeve 100″′ may have one or more of the same features as discussed with reference to the anchor sleeve 100, 100′, and/or 100″, the entire disclosure of which is incorporated herein for sake of brevity. Furthermore, the anchor sleeve 100″′ may be used in the same systems and/methods as described herein, the entire disclosure of which is incorporated herein for sake of brevity.

As previously discussed, the anchor sleeve 100″′ may have a tubular body 102″′ with a shoulder 104″′ on a proximal portion. The tubular body 102″′ may have a plurality of wings 106″′ separated or defined by one or more longitudinal slots 108″′ allowing the plurality of wings 106″′ to expand and anchor the repair strand 20 in the bone 50 (e.g., spongy cancellous bone). The tubular body 102″′ may have at least one barb 110″′ extending circumferentially around the longitudinal axis L of the tubular body 102″′. The at least one barb 110″′ may have a plurality of barbs 110″′. The at least one barb 110″′ may be formed on the plurality of wings 106″′. The at least one barb 110″′ on the plurality of wings 106″′ may be circumferentially interrupted by the one or more longitudinal slots 108″′. Additionally or alternatively, the at least one barb 110″′ may be proximal of the plurality of wings 106″′ and be uninterrupted around the circumference. As illustrated, the at least one barb 110″′ may extend substantially the entire length of the tubular body 102″′. The at least one barb 110″′ may have a distal taper to facilitate insertion and a flat proximal surface. The shoulder 104″′ may remain substantially fixed as the plurality of wings 106″′ expand.

The shoulder 104″′ may form a lip 128″′ extending at least partially around the longitudinal axis L. The shoulder 104″′ of the anchor sleeve 100″′ may have a circumferential channel 133″′. The circumferential channel 133″′ may extend at least partially around the longitudinal axis L. As illustrated, the circumferential channel 133″′ may extend less than the entire circumference of the shoulder 104″′. The circumferential channel 133″′ may be discontinuous at the lip 128″′. The lip 128″′ may have a concave upper surface 115″′ (as illustrated in FIG. 26) configured to receive the repair strand 20.

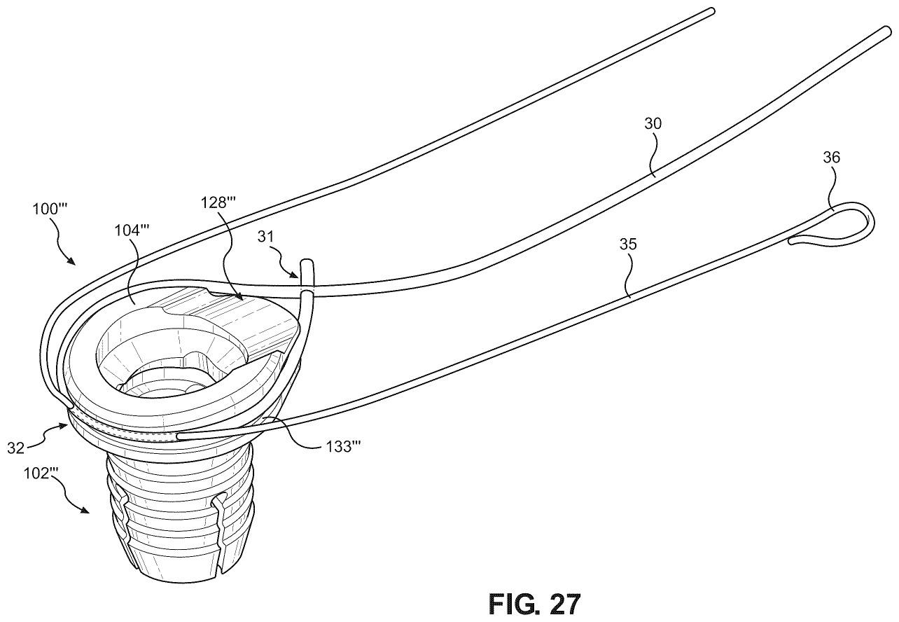

As illustrated in FIG. 27, a second repair strand 30 may be secured or fixed to the shoulder 104″′ by being received in the circumferential channel 133″′. The second repair strand 30 may secure the tissue 40 to the bone 50 as similarly discussed with reference to FIGS. 19A-20D, the entire disclosure of which is incorporated herein for sake of brevity. The second repair strand 30 may have a first end portion that is spliced, knotted, welded, or otherwise secured to itself. For example, as illustrated, the second repair strand 30 may be received in the circumferential channel 133″′ and extend through itself to secure the first end portion of the second repair strand 30 to the anchor sleeve 100″′. The second repair strand 30 may be spliced through itself at a first portion 31, such that the second repair strand 30 extends perpendicular of a longitudinal axis of the second repair strand 30. A second end portion of the second repair strand 30 may be free to be looped through the tissue 40 to secure the tissue 40. A suture threader 35 may be secured to the second repair strand 30 by extending through a longitudinal length of the second repair strand 30, for example through a second portion 32 of the second repair strand 30. The second portion 32 may be received in the circumferential channel 133″′ around the shoulder 104″′. The suture threader 35 may extend from the second repair strand 30 such that a first free end portion may be manipulated or pulled by the user and a second free end portion may have a loop 36 that may receive the second repair strand 30. In use, the free end portion of the second repair strand 30 may be passed through the tissue 40 and be threaded through the loop 36 in the suture threader 35. The suture threader 35 may then be pulled proximally from the second portion 32 to thread the free end portion of the second repair strand 30 through the second portion 32 as the loop 36 of the suture threader 35 is pulled through and proximally out of the second portion 32. The suture threader 35 may pull the free end portion of the second repair strand 30 at least partially through the circumferential channel 133″′ around the longitudinal axis L. Thus, a first end portion of the second repair strand 30 may extend through the circumferential channel 133″′ of the anchor sleeve 100″′ and be secured, for example by extending through itself at a first portion 31. A second end portion of the second repair strand 30 may be looped through the tissue 40 and secured to itself by extending through the second portion 32. The second portion 32 of the repair strand 20 may be in the circumferential channel 133″′.

FIGS. 28A-G illustrate a fifth exemplary anchor sleeve 100″″ for an anchor assembly. The anchor sleeve 100″″ may have one or more of the same features as discussed with reference to the anchor sleeve 100, 100′, 100″, and/or 100″′, the entire disclosure of which is incorporated herein for sake of brevity. Furthermore, the anchor sleeve 100″″ may be used in the same systems and/or methods as described herein, the entire disclosure of which is incorporated herein for sake of brevity.

As previously discussed, the anchor sleeve 100″″ may have a tubular body 102″″ with a shoulder 104″″ on a proximal portion. The tubular body 102″″ may have a plurality of wings 106″″ separated or defined by one or more longitudinal slots 108″″ allowing the plurality of wings 106″″ to expand and anchor the repair strand 20 in the bone 50 (e.g., spongy cancellous bone). The tubular body 102″″ may have at least one barb 110″″ extending circumferentially around the longitudinal axis L of the tubular body 102″″. The at least one barb 110″″ may have a plurality of barbs 110″″. The at least one barb 110″″ may be formed on the plurality of wings 106″″. The at least one barb 110″″ on the plurality of wings 106″″ may be circumferentially interrupted by the one or more longitudinal slots 108″″. Additionally or alternatively, the at least one barb 110″″ may be proximal of the plurality of wings 106″″ and be uninterrupted around the circumference. As illustrated, the at least one barb 110″″ may extend substantially the entire length of the tubular body 102″″. The at least one barb 110″″ may have a distal taper to facilitate insertion and a flat proximal surface. The shoulder 104″″ may remain substantially fixed as the plurality of wings 106″″ expand. The anchor sleeve 100″″ may have teeth distal of the shoulder 104″″, as discussed with reference to the previous embodiment.

The shoulder 104″″ may provide support at the edge of the bone socket for the repair strand 20 and prevent the repair strand 20 from widening the socket or cutting through bone if over-tensioned. The shoulder 104″″ may have a proximal surface 105″″ that is rounded on at least a portion of (e.g., a majority of) the proximal portion to reduce stress on any overlying tissues. The shoulder 104″″ may form a lip 128″″ extending at least partially around the longitudinal axis L. The repair strand 20 may be tensioned down onto the lip 128″″ to secure the tissue 40 to the bone 50. The lip 128″″ may have at least one recess 114″″, each configured to receive a repair strand 20 when tensioned down. As further illustrated, the lip 128″″ may have a raised partition 117″″ forming a first recess 114″″ and a second recesses 114″″ on the shoulder 104″″. Each recess 114″″ may have a surface 115″″ forming a seat for a repair strand 20, as further discussed above. Each of the surfaces 115″″ may be substantially flat and/or concave. The first recess 114″″ and the second recess 114″″ may be on the same lateral side of the shoulder 104″″ (e.g., within a 180° segment or 120° segment of the shoulder 104″″). The first recess 114″″ may receive a first repair strand 20, and the second recess 114″″ may receive a second repair strand 20, for example in a double-row bridge repair as illustrated in FIG. 14F. 19I, and/or 20D. The separation of the repair strands 20 in the recesses 114″″ may reduce friction between the repair strands 20 and/or improve alignment of the repair strands 20. The raised partition 117″″ may further reduce rotation of the anchor sleeve 100″″ relative to the bone as the anchor insert 160 is rotated into the anchor sleeve 100″″. As further illustrated in FIG. 28, the raised partition 117″″ have a height substantially equal to the proximal surface 105″″ of the shoulder 104″″. The shoulder 104″″ may have a third recess 118″″ configured to receive a protrusion of an outer sleeve 320, as discussed herein. The third recess 118″″ may be opposite of the raised partition 117″″, for example, offset by about 180° around the longitudinal axis L.

As further illustrated in FIG. 28A, D-G, the tubular body 102″″ may have a non-circular outer cross-section. The tubular body 102″″ may be inserted and/or form a corresponding non-circular socket in the bone to reduce rotation relative to the bone as the insert 160 is inserted into the anchor sleeve 100″″. The tubular body 102″″ may have outer surfaces around its perimeter with different profiles. The tubular body 102″″ may have one or more flat outer surfaces 121″″ around the perimeter of the tubular body 102″″. The one or more flat outer surfaces 121″″ may have a plurality of flat outer surfaces 121″″, such as four flat outer surfaces 121″″ as illustrated in FIG. 28D. The tubular body 102″″ may have a plurality of rounded outer surfaces 123″″ between pairs of flat outer surfaces 121″″. The longitudinal slots 108″″ may extend longitudinally through the flat outer surfaces 121″″ and the barbs 110″″ may be formed on the rounded outer surfaces 123″″. The flat outer surfaces 121″″ may not have barbs 110″″ (and be barbless) and/or the rounded outer surfaces 123″″ may not define longitudinal slots 108″″ (and be continuous). The tubular body 102″″ may be symmetric relative to one or more axis. For example, the flat outer surfaces 121″″ may be substantially the same, and the rounded outer surfaces 123″″ may be substantially the same, such that the tubular body 102″″ may be symmetric relative to at least two axis, as further illustrated in FIG. 28D. The flat outer surfaces 121″″ and/or the rounded outer surfaces 123″″ may extend a majority of the length of the tubular body 102″″. For example, the flat outer surfaces 121″″ and/or the rounded outer surfaces 123″″ may extend substantially the entire length of the tubular body 102″″. As illustrated in FIG. 28B, the anchor sleeve 100″″ may have an inner surface 120″″ defining a lumen 122″″ configured to receive the anchor insert 160. The inner surface 120″″ may have an inner thread 124″″ configured to engage the outer thread 166 of anchor insert 160 through rotation, as discussed herein.

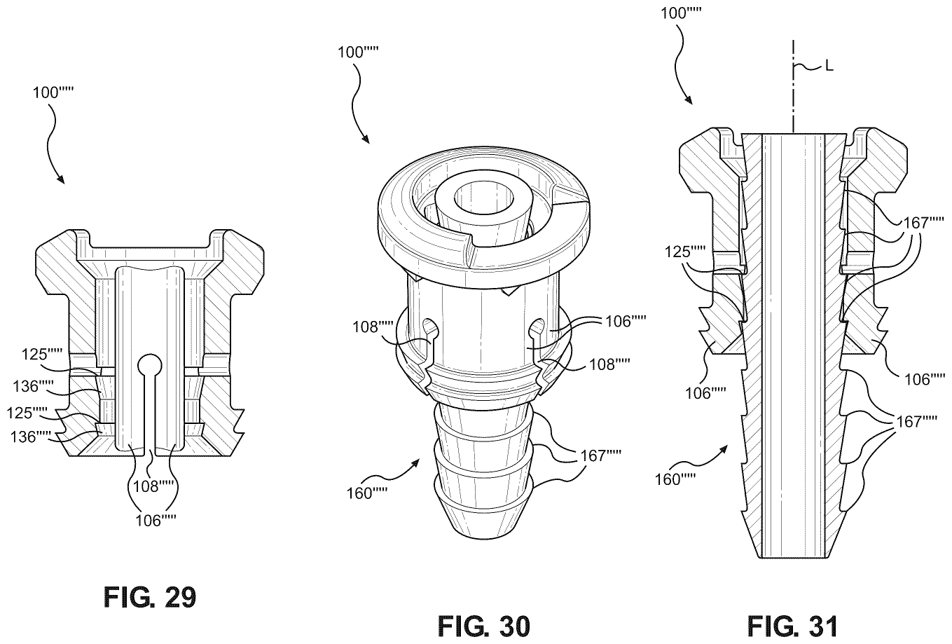

FIGS. 29-31 illustrate a sixth exemplary anchor sleeve 100″″′ and a second exemplary anchor insert 160″″′. The anchor sleeve 100″″′ and the anchor insert 160″″′ may have one or more of the same features as discussed with reference to the anchor sleeve 100, 100′, 100″, 100″′, and/or 100″″′ and anchor insert 160, the entire disclosure of which is incorporated herein for sake of brevity. Furthermore, the anchor sleeve 100″″′ and the anchor insert 160″″′ may be used in the same systems and/or methods as described herein.

However, as illustrated, the anchor insert 160″″′ may be inserted into the anchor sleeve 100″″′ with a barbed connection, instead of a threaded connection. The anchor insert 160″″′ may have an outer surface with one or more outer barbs 167″″′. The one or more barbs 167″″′ may have a distal taper to facilitate insertion and a flat proximal surface. The flat proximal surface may form a proximal portion that is radially enlarged and/or pointed. As illustrated, the anchor insert 160″″′ may have a plurality of outer barbs 167″″′ circumferentially extending around the longitudinal axis L. The plurality of barbs 167″″′ may be spaced longitudinally apart along the anchor insert 160″″′. The plurality of outer barbs 167″″′ may extend along substantially the entire length of the anchor insert 160″″′.

The anchor sleeve 100″″′ may have one or more inner barbs 125″″′ configured to engage the one or more outer barbs 167″″′. As illustrated, the one or more inner barbs 125″″′ may be formed on an inner surface of the anchor sleeve 100″″′ by one or more recesses 136″″′ configured to receive the one or more outer barbs 167″″′. As further illustrated, the one or more inner barbs 125″″′ may include a plurality of inner barbs 125″″′ circumferentially extending around the longitudinal axis L. The one or more inner barbs 125″″′ may be on an inner surface of a plurality of wings 106″″′ and be interrupted by one or more longitudinal slots 108″″′.

It will also be appreciated by those skilled in the art that modifications can be made to the example embodiments described herein without departing from the invention. Structural features of systems and apparatuses described herein can be replaced with functionally equivalent parts or omitted entirely. Moreover, it will be appreciated that features from the embodiments can be combined with each other without departing from the disclosure.

What is claimed is:

1. An anchor assembly configured to secure a tissue to a bone using a repair strand, the anchor assembly comprising:
an anchor sleeve comprising a plurality of wings configured to expand, the anchor sleeve defining a lumen, and the anchor sleeve being configured to be inserted into a bone socket in the bone;
an anchor tip having an opening configured to receive the repair strand; and
an anchor insert configured to be inserted into the lumen of the anchor sleeve, and the anchor insert being configured to expand the plurality of wings of the anchor sleeve while the anchor insert is inserted into the lumen of the anchor sleeve with the repair strand between the anchor insert and the anchor sleeve.

2. The anchor assembly of claim 1, wherein the anchor sleeve comprises an inner thread configured to engage an outer thread of the anchor insert.

3. The anchor assembly of claim 1, wherein the anchor insert is configured to rotate relative to the anchor tip about a longitudinal axis while being inserted into the anchor sleeve.

4. The anchor assembly of claim 1, wherein the anchor insert is proximal of the anchor tip along a drive shaft.

5. The anchor assembly of claim 1, wherein a proximal portion of the anchor sleeve has a shoulder.

6. The anchor assembly of claim 5, wherein the anchor sleeve has a plurality of teeth distal of the shoulder and configured to reduce rotation of the anchor sleeve relative to the bone.

7. The anchor assembly of claim 5, wherein the shoulder has a proximal surface with a recess configured to receive the repair strand.

8. The anchor assembly of claim 7, wherein the anchor sleeve has an inner surface with at least one longitudinal channel configured to receive the repair strand, wherein the at least one longitudinal channel interrupts an inner thread of the anchor sleeve.

9. The anchor assembly of claim 8, wherein the at least one longitudinal channel is at least partially in a common plane as the recess.

10. The anchor assembly of claim 5, wherein the shoulder has a lip extending radially of a tubular body.

11. An anchor sleeve comprising:
a tubular body configured to be inserted into a socket of a bone, wherein the tubular body has a lumen configured to receive an anchor insert, and the tubular body has a plurality of wings configured to expand; and
a shoulder extending at least partially around the lumen, wherein the shoulder is configured to be seated on an outer surface of the bone, the shoulder has a lip extending radially of the tubular body, and the lip is configured to support a repair strand extending from a tissue and through the lumen of the tubular body.

12. The anchor sleeve of claim 11, wherein the lip extends further radially than a portion of the shoulder.

13. The anchor sleeve of claim 11, wherein the tubular body has an inner thread configured to engage an outer thread of the anchor insert.

14. The anchor sleeve of claim 11, wherein the tubular body has an inner surface with at least one longitudinal channel configured to receive the repair strand.

15. The anchor sleeve of claim 14, wherein the at least one longitudinal channel is at least partially in a common plane as the lip.

16. The anchor sleeve of claim 11, wherein the tubular body has a plurality of barbs on an outer surface.

17. The anchor sleeve of claim 11, further comprising a suture threader configured to secure an end portion of a second repair strand to the shoulder.

18. An anchor sleeve comprising:
a tubular body configured to be inserted into a socket of a bone, wherein the tubular body has a lumen configured to receive an anchor insert, and the tubular body has a plurality of wings configured to expand; and
a shoulder extending at least partially around the lumen, wherein the shoulder is configured to be seated on an outer surface of the bone, the shoulder has a raised protrusion defining a first recess and a second recess, the first recess is configured to receive a first repair strand, and the second recess is configured to receive a second repair strand.

19. The anchor sleeve of claim 18, wherein the shoulder has a lip, and the raised protrusion is on the lip.

20. The anchor sleeve of claim 19, wherein the lip has a first proximal surface in the first recess and a second proximal surface in the second recess.
